SEQUENCE LISTING (14 sequences)

SEQ ID NO: 1 - DNA, 5804 bp, Artificial Sequence
  Description of Artificial Sequence; Note = synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcacgctgccgcaagcactcagggcgcaagggctcgtaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtgggaggctattcggcttgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtgctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgccccagcgcatcgctcggcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcggggactctggggttcgtactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatcagtggtggcgacaggactataaagataccaggcgtttcccctggcggctccctcgtgcgctctcctgttcctgcctttcggtttccggtgtcattccgctgttatggccgcgtttgtctcattccacgcctgacactcagttccgggtaggcagttcgctccaagctggactgtatgcacgaaccccccgttcagtccgaccgctgcgccttatccggtaacttcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaaggattacgcgcagaccaacgtctcaagaagatcatcttattaatcagataaaatcgaaatgaccgaccaagcgacgcccacctgcctcacgagtttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatccttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 5412 bp, Artificial Sequence
  Description of Artificial Sequence; Note = synthetic construct
gaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccgatccggtggtggtgcaaatcaaagaactgctcctcagtggatgttgcctttacttctaggcctgtacggaagtgttacttctgctctaaaagctgcggaattgtacccgcggataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatctcaaagaggtccgacgcgtatgtgccgcaatatatatgacccacttttatgcttcaaactattttttactgatgagataatttcggaaattgtaaaatggacaaatgctgagatatcattgaaacgtcgggaatctatgacaggtgctacatttcgtgacacgaatgaagatgaaatctatgctttctttggtattctggtaatgacagcagtgagaaaagataaccacatgtccacagatgacctctttgatcgatctttgtcaatggtgtacgtctctgtaatgagtcgtgatcgttttgattttttgatacgatgtcttagaatggatgacaaaagtatacggcccacacttcgagaaaacgatgtatttactcctgttagaaaaatatgggatctctttatccatcagtgcatacaaaattacactccaggggctcatttgaccatagatgaacagttacttggttttagaggacggtgtccgtttaggatgtatatcccaaacaagccaagtaagtatggaataaaaatcctcatgatgtgtgacagtggtacgaagtatatgataaatggaatgccttatttgggaagaggaacacagaccaacggagtaccactcggtgaatactacgtgaaggagttatcaaagcctgtgcacggtagttgtcgtaatattacgtgtgacaattggttcacctcaatccctttggcaaaaaacttactacaagaaccgtataagttaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggttcaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattaggatctaagctgcaataaacaagttaacaacaacaattgcattcattttatgtttcaggttcagggggaggtgtgggaggttttttcggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 3 - DNA, 8551 bp, Artificial Sequence
  Description of Artificial Sequence; Note = synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcacgctgccgcaagcactcagggcgcaagggctcgtaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtgggaggctattcggcttgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtgctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgccccagcgcatcgctcggcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcggggactctggggttcgtactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatcagtggtggcgacaggactataaagataccaggcgtttcccctggcggctccctcgtgcgctctcctgttcctgcctttcggtttccggtgtcattccgctgttatggccgcgtttgtctcattccacgcctgacactcagttccgggtaggcagttcgctccaagctggactgtatgcacgaaccccccgttcagtccgaccgctgcgccttatccggtaacttcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaaggattacgcgcagaccaacgtctcaagaagatcatcttattaatcagataaaatcgaaatgaccgaccaagcgacgcccacctgcctcacgagtttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatccttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatctagagtcgacctgcaggtcgactctagaggatccgaaaaaacctcccacacctccccctgaacctgaaacataaaatgaatgcaattgttgttgttaacttgtttattgcagcttagatcctaattagcttaacttatacataatagaaacaaattatacttattagtcagtcagaaacaactttggcacatatcaatattatgctctcgacaaataacttttttgcattttttgcacgatgcatttgcctttcgccttattttagaggggcagtaagtacagtaagtacgttttttcattactggctcttcagtactgtcatctgatgtaccaggcacttcatttggcaaaatattagagatattatcgcgcaaatatctcttcaaagtaggagcttctaaacgcttacgcataaacgatgacgtcaggctcatgtaaaggtttctcataaattttttgcgactttggaccttttctcccttgctactgacattatggctgtatataataaaagaatttatgcaggcaatgtttatcattccgtacaataatgccataggccacctattcgtcttcctactgcaggtcatcacagaacacatttggtctagcgtgtccactccgcctttagtttgattataatacataaccatttgcggtttaccggtactttcgttgatagaagcatcctcatcacaagatgataataagtataccatcttagctggcttcggtttatatgagacgagagtaaggggtccgtcaaaacaaaacatcgatgttcccactggcctggagcgactgtttttcagtacttccggtatctcgcgtttgtttgatcgcacggttcccacaatggttaacttatacggttcttgtagtaagttttttgccaaagggattgaggtgaaccaattgtcacacgtaatattacgacaactaccgtgcacaggctttgataactccttcacgtagtattcaccgagtggtactccgttggtctgtgttcctcttcccaaataaggcattccatttatcatatacttcgtaccactgtcacacatcatgaggatttttattccatacttacttggcttgtttgggatatacatcctaaacggacaccgtcctctaaaaccaagtaactgttcatctatggtcaaatgagcccctggagtgtaattttgtatgcactgatggataaagagatcccatatttttctaacaggagtaaatacatcgttttctcgaagtgtgggccgtatacttttgtcatccattctaagacatcgtatcaaaaaatcaaaacgatcacgactcattacagagacgtacaccattgacaaagatcgatcaaagaggtcatctgtggacatgtgrttatcttttctcactgctgtcattaccagaataccaaagaaagcatagatttcatcttcattcgtgtcacgaaatgtagcacctgtcatagattcccgacgtttcaatgatatctcagcatttgtccattttacaatttccgaaattatctcatcagtaaaaaatagtttgaagcataaaagtgggtcatatatattgcggcacatacgcgtcggacctctttgagatctgacaatgttcagtgcagagactcggctacgcctcgtggactttgaagttgaccaacaatgtttattcttacctctaatagtcctctgtggcaaggtcaagattctgttagaagccaatgaagaacctggttgttcaataacattttgttcgtctaatatttcactaccgcttgacgttggctgcacttcatgtacctcatctataaacgcttcttctgtatcgctctggacgtcatcttcacttacgtgatctgatatttcactgtcagaatcctcaccaacaagctcgtcatcgctttgcagaagagcagagaggatatgctcatcgtctaaagaactacccattttatccgcgggtacaattccgcagcttttagagcagaagtaacacttccgtacaggcctagaagtaaaggcaacatccactgaggagcagttctttgatttgcaccaccaccggatcgggacctgaaataaaagacaaaaagactaaacttaccagttaactttctggtttttcagttcctcgagtaccggatcctctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 311 bp, Artificial Sequence
  Description of Artificial Sequence; Note = synthetic construct
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagata

SEQ ID NO: 5 - DNA, 236 bp, Artificial Sequence
  Description of Artificial Sequence; Note = synthetic construct
ttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaa

SEQ ID NO: 6 - DNA, 1785 bp, Artificial Sequence
  Description of Artificial Sequence; Note = synthetic construct
atgggatcatctctggacgacgagcacatcctgtctgctctgctgcagagtgatgacgagctggtgggagaggactccgattcagagatctccgaccatgtgagtgaggatgacgtccagtcagacacagaagaggctttcattgatgaggtccacgaagtgcagcccacctcaagtggatcagagattctggacgagcagaacgtgattgaacagcctgggagcagtctggcctcaaacaggattctgacactgccacagcggaccattcgcggcaaaaacaaacattgctggagcacaagtaaatccaccagacgaagccgggtgtcagccctgaatattgtgcgcagccagcggggccccaccaggatgtgtcgaaacatctacgatcctctgctgtgtttcaagctgttcttcaccgatgagattatttcagaaatcgtgaagtggaccaacgcagaaatcagcctgaaacggcgcgagtcaatgaccggcgccacctttagagatacaaatgaggatgagatctacgcattctttggaattctggtcatgaccgcagtcagaaaggataaccatatgagtacagacgacctgttcgaccggagcctgtccatggtctatgtgagtgtgatgtctcgggataggttcgactttctgatccgctgcctgcgaatggacgataagagtatcagacctacactgcgggaaaacgacgtctttacccccgtgcgaaagatttgggacctgtttatccaccagtgtattcagaactatacacccggcgcccatctgaccattgacgaacagctgctgggcttcaggggcagatgccccttccgcatgtacatcccaaacaagcccagcaaatatggcattaagatcctgatgatgtgcgacagcggcaccaagtacatgatcaatggaatgccttacctggggcgcggcactcagacaaatggcgtccctctgggagagtactacgtcaaggaactgagcaaacccgtccacgggtcatgtcggaacatcacctgcgacaactggttcacctccattccactggctaagaacctgctgcaggagccctacaaactgacaatcgtgggcacagtgagatctaacaagagagagatcccagaggtgctgaagaattctcggtctaggcccgtgggcacttcaatgttttgctttgatggcccactgacactggtctcctacaagccaaagcctgcaaagatggtgtatctgctgagttcctgtgatgaagacgcctccattaatgaaagcaccggcaaacctcagatggtcatgtattacaaccagaccaaaggaggggtcgacaccctggatcagatgtgttccgtgatgacatgtagcagaaaaaccaatcgctggcctatggctctgctgtatggcatgatcaacatcgcatgcatcaacagcttcattatctactcacacaatgtgtcaagcaaaggcgagaaagtgcagagccgcaaaaaattcatgaggaacctgtacatgtccctgacttcttcctttatgaggaagcggctggaagctcccacactgaagcgctacctgcgcgataacattagtaacatcctgcccaacgaagtgcctggaacttccgatgatagcaccgaagaacctgtgatgaagaagagaacatactgcacatattgcccttcaaaaattcggcggaaggcaaatgcaagctgcaagaagtgcaagaaagtgatctgccgggagcacaacatcgatatgtgtcagagctgcttttga

SEQ ID NO: 7 - DNA, 7121 bp, Artificial Sequence
  Description of Artificial Sequence; Note = synthetic construct
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagctcaagcttcgaattctgcagtcgacggtaccgcgggcccgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtctatctacatggccaagaagcccgtgcagctgcccggctactactacgtggactccaagctggacatcacctcccacaacgaggactacaccatcgtggagcagtacgagcgcgccgagggccgccaccacctgttcctgtagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggaggcctaggcttttgcaaaaagcttgattcttctgacacaacagtctcgaacttaaggctagagaattcatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaccgcggctctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggattttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatctttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagataagatct

SEQ ID NO: 8 - DNA, 7165 bp, Artificial Sequence
  Description of Artificial Sequence; Note = synthetic construct
tcgagctcaagcttcgaattctgcagtcgacggtaccgcgggcccgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggaggcctaggcttttgcaaaaagcttgattcttctgacacaacagtctcgaacttaaggctagagaattcatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaccgcggctctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggattttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatctttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagataagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcc

SEQ ID NO: 9 - DNA, 8144 bp, Artificial Sequence
  Description of Artificial Sequence; Note = synthetic construct
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggagcgcgtcatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaaccctgtgcaacagccactacattacttcaaactgagatccttccttttgagggagcaagtccttccctttcattttttccagtcttcctccctgtgtattcattctcatgattattattttagtgggggcggggtgaattcacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacatgcacagagatgcctggctacctcgccctgccttcagcctcacggggctcagtctctttttctctttggtgccaccaggacggagcatggaggtcacagtacctgccaccctcaacgtcctcaatggctctgacgcccgcctgccctgcaccttcaactcctgctacacagtgaaccacaaacagttctccctgaactggacttaccaggagtgcaacaactgctctgaggagatgttcctccagttccgcatgaagatcattaacctgaagctggagcggtttcaagaccgcgtggagttctcagggaaccccagcaagtacgatgtgtcggtgatgctgagaaacgtgcagccggaggatgaggggatttacaactgctacatcatgaacccccctgaccgccaccgtggccatggcaagatccatctgcaggtcctcatggaagagccccctgagcgggactccacggtggccgtgattgtgggtgcctccgtcgggggcttcctggctgtggtcatcttggtgctgatggtggtcaagtgtgtgaggagaaaaaaagagcagaagctgagcacagatgacctgaagaccgaggaggagggcaagacggacggtgaaggcaacccggatgatggtgccaagtaggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggattttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatctttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagataagatct

SEQ ID NO: 10 - DNA, 7520 bp, Artificial Sequence
  Description of Artificial Sequence; Note = synthetic construct
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggagcgcgtcatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaaccctgtgcaacagccactacattacttcaaactgagatccttccttttgagggagcaagtccttccctttcattttttccagtcttcctccctgtgtattcattctcatgattattattttagtgggggcggggtgaattcacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacatgcacagagatgcctggctacctcgccctgccttcagcctcacggggctcagtctctttttctctttggtgccaccaggacggagcatggaggtcacagtacctgccaccctcaacgtcctcaatggctctgacgcccgcctgccctgcaccttcaactcctgctacacagtgaaccacaaacagttctccctgaactggacttaccaggagtgcaacaactgctctgaggagatgttcctccagttccgcatgaagatcattaacctgaagctggagcggtttcaagaccgcgtggagttctcagggaaccccagcaagtacgatgtgtcggtgatgctgagaaacgtgcagccggaggatgaggggatttacaactgctacatcatgaacccccctgaccgccaccgtggccatggcaagatccatctgcaggtcctcatggaagagccccctgagcgggactccacggtggccgtgattgtgggtgcctccgtcgggggcttcctggctgtggtcatcttggtgctgatggtggtcaagtgtgtgaggagaaaaaaagagcagaagctgagcacagatgacctgaagaccgaggaggagggcaagacggacggtgaaggcaacccggatgatggtgccaagtaggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggattttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatctttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagataagatct

SEQ ID NO: 11 - DNA, 7756 bp, Artificial Sequence
  Description of Artificial Sequence; Note = synthetic construct
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagatggggaggctgctggccttagtggtcggcgcggcactggtgtcctcagcctgcgggggctgcgtggaggtggactcggagaccgaggccgtgtatgggatgaccttcaaaattctttgcatctcctgcaagcgccgcagcgagaccaacgctgagaccttcaccgagtggaccttccgccagaagggcactgaggagtttgtcaagatcctgcgctatgagaatgaggtgttgcagctggaggaggatgagcgcttcgagggccgcgtggtgtggaatggcagccggggcaccaaagacctgcaggatctgtctatcttcatcaccaatgtcacctacaaccactcgggcgactacgagtgccacgtctaccgcctgctcttcttcgaaaactacgagcacaacaccagcgtcgtcaagaagatccacattgaggtagtggacaaagccaacagagacatggcatccatcgtgtctgagatcatgatgtatgtgctcattgtggtgttgaccatatggctcgtggcagagatgatttactgctacaagaagatcgctgccgccacggagactgctgcacaggagaatgcctcggaatacctggccatcacctctgaaagcaaagagaactgcacgggcgtccaggtggccgaatagacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacatgcacagagatgcctggctacctcgccctgccttcagcctcacggggctcagtctctttttctctttggtgccaccaggacggagcatggaggtcacagtacctgccaccctcaacgtcctcaatggctctgacgcccgcctgccctgcaccttcaactcctgctacacagtgaaccacaaacagttctccctgaactggacttaccaggagtgcaacaactgctctgaggagatgttcctccagttccgcatgaagatcattaacctgaagctggagcggtttcaagaccgcgtggagttctcagggaaccccagcaagtacgatgtgtcggtgatgctgagaaacgtgcagccggaggatgaggggatttacaactgctacatcatgaacccccctgaccgccaccgtggccatggcaagatccatctgcaggtcctcatggaagagccccctgagcgggactccacggtggccgtgattgtgggtgcctccgtcgggggcttcctggctgtggtcatcttggtgctgatggtggtcaagtgtgtgaggagaaaaaaagagcagaagctgagcacagatgacctgaagaccgaggaggagggcaagacggacggtgaaggcaacccggatgatggtgccaagtaggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggaggcctaggcttttgcaaaaagcttgattcttctgacacaacagtctcgaacttaaggctagagaattcatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaccgcggctctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggattttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatctttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagataagatct

SEQ ID NO: 12 - DNA, 7948 bp, Artificial Sequence
  Description of Artificial Sequence; Note = synthetic construct
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagatggggaggctgctggccttagtggtcggcgcggcactggtgtcctcagcctgcgggggctgcgtggaggtggactcggagaccgaggccgtgtatgggatgaccttcaaaattctttgcatctcctgcaagcgccgcagcgagaccaacgctgagaccttcaccgagtggaccttccgccagaagggcactgaggagtttgtcaagatcctgcgctatgagaatgaggtgttgcagctggaggaggatgagcgcttcgagggccgcgtggtgtggaatggcagccggggcaccaaagacctgcaggatctgtctatcttcatcaccaatgtcacctacaaccactcgggcgactacgagtgccacgtctaccgcctgctcttcttcgaaaactacgagcacaacaccagcgtcgtcaagaagatccacattgaggtagtggacaaagccaacagagacatggcatccatcgtgtctgagatcatgatgtatgtgctcattgtggtgttgaccatatggctcgtggcagagatgatttactgctacaagaagatcgctgccgccacggagactgctgcacaggagaatgcctcggaatacctggccatcacctctgaaagcaaagagaactgcacgggcgtccaggtggccgaatagacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacatgcacagagatgcctggctacctcgccctgccttcagcctcacggggctcagtctctttttctctttggtgccaccaggacggagcatggaggtcacagtacctgccaccctcaacgtcctcaatggctctgacgcccgcctgccctgcaccttcaactcctgctacacagtgaaccacaaacagttctccctgaactggacttaccaggagtgcaacaactgctctgaggagatgttcctccagttccgcatgaagatcattaacctgaagctggagcggtttcaagaccgcgtggagttctcagggaaccccagcaagtacgatgtgtcggtgatgctgagaaacgtgcagccggaggatgaggggatttacaactgctacatcatgaacccccctgaccgccaccgtggccatggcaagatccatctgcaggtcctcatggaagagccccctgagcgggactccacggtggccgtgattgtgggtgcctccgtcgggggcttcctggctgtggtcatcttggtgctgatggtggtcaagtgtgtgaggagaaaaaaagagcagaagctgagcacagatgacctgaagaccgaggaggagggcaagacggacggtgaaggcaacccggatgatggtgccaagtaggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggattttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatctttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagataagatct

SEQ ID NO: 13 - DNA, 9314 bp, Artificial Sequence
  Description of Artificial Sequence; Note = synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcacgctgccgcaagcactcagggcgcaagggctcgtaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtgggaggctattcggcttgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtgctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgccccagcgcatcgctcggcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcggggactctggggttcgtactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatcagtggtggcgacaggactataaagataccaggcgtttcccctggcggctccctcgtgcgctctcctgttcctgcctttcggtttccggtgtcattccgctgttatggccgcgtttgtctcattccacgcctgacactcagttccgggtaggcagttcgctccaagctggactgtatgcacgaaccccccgttcagtccgaccgctgcgccttatccggtaacttcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaaggattacgcgcagaccaacgtctcaagaagatcatcttattaatcagataaaatcgaaatgaccgaccaagcgacgcccacctgcctcacgagtttcgattccaccgccgccttctatgaaaggttgggcttcggaatcgttttccgggacgccggctggatgatcctccagcgcggggatctcatgctggagttcttcgcccaccccaacttgtttattgcagcttactcttacgcggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagcccgggctcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 7827 bp, Artificial Sequence
  Description of Artificial Sequence; Note = synthetic construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagctgatccactagtccagtgtggtggaattcgctagcgccaccatggcccccaagaagaagaggaaggtgggaatccatggggtacccgccatggcggagaggcccttccagtgtcgaatctgcatgcgtaacttcagtcgtagtgaccacctgagccggcacatccgcacccacacaggcgagaagccttttgcctgtgacatttgtgggaggaaatttgccgacaaccgggaccgcacaaagcataccaagatacacacgggcggacagcggccgtacgcatgccctgtcgagtcctgcgatcgccgcttttctgacaggaagacacttatcgagcatatccgcatccacaccggtcagaagcccttccagtgtcgaatctgcatgcgtaacttcagtaccagcagcggcctgagccgccacatccgcacccacacaggatctcagaagcccttccagtgtcgaatctgcatgcgtaacttcagtcgtagtgaccacctgagcgaacacatccgcacccacacaggcgagaagccttttgcctgtgacatttgtgggaggaaatttgccaccagcagcgaccgcacaaagcataccaagatacacctgcgccaaaaagatgcggcccggggatccggcggctctggaggttccggaggctctggtggttctggaactagtatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatctcaaagaggtccgacgcgtatgtgccgcaatatatatgacccacttttatgcttcaaactattttttactgatgagataatttcggaaattgtaaaatggacaaatgctgagatatcattgaaacgtcgggaatctatgacaggtgctacatttcgtgacacgaatgaagatgaaatctatgctttctttggtattctggtaatgacagcagtgagaaaagataaycacatgtccacagatgacctctttgatcgatctttgtcaatggtgtacgtctctgtaatgagtcgtgatcgttttgattttttgatacgatgtcttagaatggatgacaaaagtatacggcccacacttcgagaaaacgatgtatttactcctgttagaaaaatatgggatctctttatccatcagtgcatacaaaattacactccaggggctcatttgaccatagatgaacagttacttggttttagaggacggtgtccgtttaggatgtatatcccaaacaagccaagtaagtatggaataaaaatcctcatgatgtgtgacagtggtacgaagtatatgataaatggaatgccttatttgggaagaggaacacagaccaacggagtaccactcggtgaatactacgtgaaggagttatcaaagcctgtgcacggtagttgtcgtaatattacgtgtgacaattggttcacctcaatccctttggcaaaaaacttactacaagaaccgtataagttaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactcgagtctagctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctga

(19) United States
(12) Patent Application Publication
(10) Pub. No.: US 2009/0042297 A1
(43) Pub. Date: Feb. 12, 2009

(54) Piggybac transposon-based vectors and methods of nucleic acid integration

(76) Inventors: Alfred L. George, JR., Brentwood, TN (US); Matthew H. Wilson, Pearland, TX (US); Kristopher M. Kahlig, Nashville, TN (US)

Correspondence Address: Ballard Spahr Andrews & Ingersoll, LLP, SUITE 1000, 999 PEACHTREE STREET, ATLANTA, GA, 30309-3915, US

(21) Appl. No.: 12/156,531

(22) Filed: Jun. 2, 2008

Related U.S. Application Data
(60) Provisional application No. 60/932,726, filed on Jun. 1, 2007.

(51) Int. Cl. C12N 15/87; C12N 15/11

(52) U.S. Cl. 435/455; 536/23.2

(57) ABSTRACT

Disclosed herein are compositions comprising integrating enzymes that can deliver nucleic acids to a target DNA. Additionally, the methods of using the compositions disclosed herein relate to treatments for a variety of infections, conditions, and genetic disorders.

### I. CROSS-REFERENCE TO RELATED APPLICATIONS

This application claims the benefit of U.S. provisional application No. 60/932,726 filed on Jun. 1, 2007. The aforementioned application is herein incorporated by this reference in its entirety.

### II. STATEMENT OF FEDERALLY SPONSORED RESEARCH

This invention was made with partial government support under DOD grant F49620-01-1-0429. The United States government has certain rights in the invention.

### III. BACKGROUND OF THE INVENTION

Transposon systems have been harnessed for non-viral gene delivery and show promise for potential gene therapy applications in humans. Currently, the most widely used transposon system for pre-clinical gene therapy studies is Sleeping Beauty (SB), a member of the Tc1/mariner family of transposable elements resurrected from the fish genome [1]. Much effort has been applied toward evaluating and improving SB transposition including mutagenesis to create more active transposons [2-5], the use of RNA to deliver the transposase enzyme [6], and mapping of integration sites in human cells to evaluate safety of SB transposition into the human genome [7]. However, SB transposition, like other members of the Tc1/mariner family [8,9], is limited by overproduction inhibition which occurs with increasing transposase expression [3, 5, 10]. This phenomenon can be detrimental to gene transfer efficiency in cultured cells and in vivo [11, 12].

The piggyBac system, derived from the cabbage looper moth Trichoplusia ni, represents an alternative transposon for gene delivery into mammalian cells. These transposable elements were initially discovered in mutant Baculovirus strains hence their name “piggyBac” [13-15]. The original piggyBac element is ˜2.4 kb with identical 13 base pair (bp) terminal inverted repeats and additional asymmetric 19 bp internal repeats [16-18]. The piggyBac element can be divided to insert a transgene between the inverted repeat elements and elements and transposition activity enabled by providing the piggyBac transposase enzyme from a separate vector. This arrangement permits a “cut and paste” mediated transposition of a transgene into the genome at TTAA nucleotide elements [13, 19]. PiggyBac was recently observed capable of delivering large (9.1-14.3 kb) transposable elements without a significant reduction in efficiency [20]. However, piggyBac transposition has not been well characterized in human cells.

Before the piggyBac system can be considered as a delivery method for gene therapy in man, a more detailed study of its activity in human cells is necessary. The present disclosure shows that piggyBac is highly efficient and has specific advantages including loss of overproduction inhibition and precise excision in human cells. PiggyBac exhibits advantageous properties compared with SB in mediating gene transfer in human cells.

### IV. SUMMARY OF THE INVENTION

In accordance with the purposes of this invention, as embodied and broadly described herein, this invention, in one aspect, relates to non-viral vectors for integration of transgenes into the genome of a subject and methods of their use.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### V. BRIEF DESCRIPTION OF THE DRAWINGS

The accompanying drawings, which are incorporated in and constitute a part of this specification, illustrate several embodiments of the invention and together with the description, serve to explain the principles of the invention. Specific examples of the invention are seen in the Examples.

FIG. 1 shows that piggyBac exhibits efficient transposition in human cells. A, schematic of transposase and transposon constructs. CMV, immediate early CMV promoter; intron, SV40 intron; pA, polyadenylation sequence; SB12, hyperactive SB transposase; pT3, hyperactive SB transposon with identical IR elements; Kan/Neo, kanamycin/neomycin resistance resistance cassette; p15A, origin of replication; SB IR elements are shaded; piggyBac IR elements are hatched. B and C, transposition assays comparing SB12 to piggyBac (N=3±SEM). HEK293 or HeLa cells were transfected with transposase (400 ng) and transposon (2 μg) plasmids, passaged into G418-containing media, and selected for 2 weeks as described in Materials and Methods. *=p<0.05 by two way ANOVA comparing piggyBac transposition to that of SB12. #=p<0.05 by two way ANOVA comparing HEK-293 cells to HeLa cells for the given transposase.

FIG. 2 shows an Excision assay of SB and piggyBac. Three days after HEK293 cells were transfected, plasmid DNA was isolated and used as a template for PCR to amplify from plasmids which have undergone excision of the transposon segment and repair (representative data from one of three experiments are illustrated).

FIG. 3 shows a Sequence logo analysis of piggyBac and SB integration sites. Weblogo was used to analyze known piggyBac integration sites for possible consensus target sites for integration. Shown are the determined consensus logo (A) and frequency (B) plots from integration sites determined as described herein.

FIG. 4 shows that PiggyBac lacks overproduction inhibition. The presence or absence of overproduction inhibition of piggyBac (with pTpB) and SB12 (with pT3) were evaluated at 2 μg (A), 200 ng (B), and 50 ng (C) of transposon DNA with increasing amounts of transposase transfected in HEK293 cells (N=3±SEM). DNA was kept constant throughout all transfections using non-recombinant pIRESpuro3 plasmid. D, the maximal activity of piggyBac was compared to SB12 at the varying transposon DNA amounts (N=3±SEM). E, Western analysis of SB12 and HA-piggyBac illustrating increased transposase expression with increased transfected transposase DNA (representative data from one of three experiments). Cells were transfected with equivalent DNA amounts exactly as in the overproduction inhibition assays. Each lane was loaded with 15 μg of protein lysate. *=p<0.05 comparing piggyBac to SB12 at the transfected transposase DNA amount (A-C) or maximal activity at the given transposon DNA amount (D).

FIG. 5 shows a helper-independent piggyBac transposase-transposon with enhanced activity in human cells. A, schematic of helper-independent vectors with components as described in FIG. 1A. B, transposition assays of helper-independent vectors in HEK293 cells. Shaded bars represent transfections with the transposase (1 μg) and transposon (1 μg) supplied separately on two different plasmids. Open bars represent transfections with helper-independent vectors (1 μg of helper independent vector with 1 μg of pIRESpuro3 to keep DNA amount of pIRESpuro3 to keep DNA amount constant). N=3±SEM. *=p<0.05 comparing PB+pTpB to SB12+pT3, and **=p<0.05 comparing pPB-Nori to PB+pTpB. Statistical analysis was performed using ANOVA followed by a Bonferroni post test comparison.

TABLE 1 shows the frequencies of piggyBac integration events within intragenic regions of human cells.

TABLE 2 shows PiggyBac integration frequencies into genomic repeat elements.

FIG. 6 shows ZFP addition to piggyBac does not alter its activity. The CH₂K-zinc finger protein (CH₂K-ZFP, described in innovation #1) was added to the N-terminus of SB12 (a hyperactive version) and piggyBac and activity was quantitated using a colony count assay in a similar experimental protocol as FIGS. 1B and 1C.

FIG. 7 shows Southern analysis of HEK293 cell clones derived from piggyBac gene transfer. Southern blot was used to determine the number of neomycin resistance genes integrated into clonal cells (derived from one cell) revealing >15 integrations per cell for the representative 4 cell lines shown

FIG. 8 shows simultaneous multi gene transfer using piggyBac. Cells were selected for neomycin resistance after transfection of a neomycin resistance transposon and a luciferase plasmid (above panel) or a neomycin resistance transposon and a luciferase transposon (lower panel). Cells were then evaluated for luciferase activity revealing multi-transposon-gene integration (lower panel). HEK-293 (1×10⁶) were transiently transfected with plasmid DNA (500 ng of pCMV-piggyBac, 1 μg of pTpB and 1 μg of pT-CAGLuc) using FuGENE®6 (Roche Diagnostics, Indianapolis, Ind.). Two days post transfection, cells were split (1:600 dilution) and placed in media containing 800 μg/mL G418. After 2 weeks of G418 selection, colonies of cells within 100 mm dishes were washed in phosphate buffered saline. Cells were then incubated at 37 degrees C. for 5 minutes in 150 mg/ml luciferin substrate (Xenogen, Inc., Cranbury, N.J.) in PBS. Luciferase expression was detected by imaging plates of cells using a Bio-Rad Chemidoc XRS System with a 5 minute exposure time.

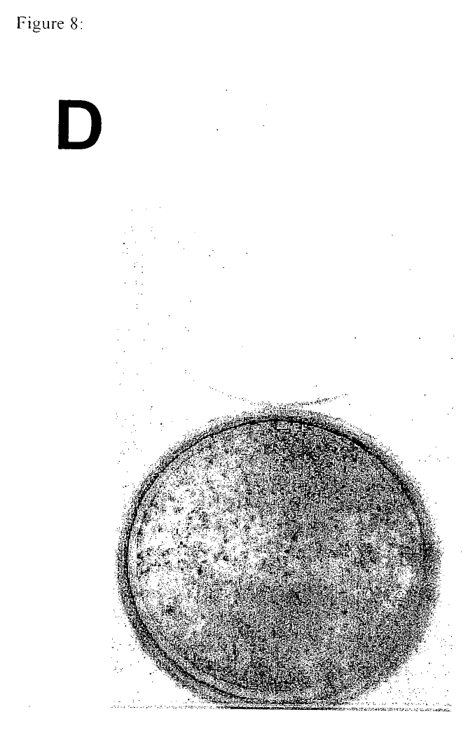

FIG. 9 shows an example of using piggyBac to simultaneously integrate 4 different genes into a cell type of interest.

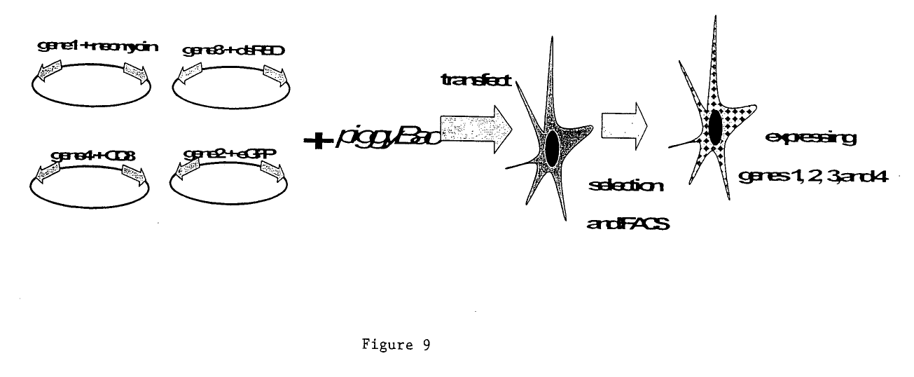

FIG. 10 shows a schematic representation of the piggyBac multi-gene transfer vectors.

FIG. 11 shows the stable integration of simultaneous multiple transgenes using the piggyBac transposon system. A, design of two multi-cistronic piggyBac transposon vectors. Top construct (10.9 kb transposon) encodes the human voltage-gated sodium channel SCN1A fused to the fluorescent protein Venus (SCN1A-Venus) driven by the CMV immediate early promoter, and the gene (Neo/Kan) encoding resistance to the aminoglycoside antibiotics neomycin and kanamycin driven by the SV40 promoter. The lower construct (5.8 kb transposon) encodes two human voltage-gated sodium channel accessory subunits (SCN1B, SCN2B) separated by a viral internal ribosome entry sequence (IRES) driven by the CMV promoter, and a puromycin resistance gene (Puro) driven by the SV40 promoter. B, photograph of methylene blue stained 100 mm tissue culture dishes 3 weeks after transfection of HEK-293 cells with both transposons and dual selection with puromycin and G418 (neomycin substitute). The left dish labeled “−transposase” was not co-transfected with the piggyBac transposase plasmid. The right dish labeled “+transposase” was co-transfected with the piggyBac transposase plasmid. Only cells co-transfected with transposase acquired dual antibiotic resistance. C, representative whole-cell patch-clamp recording of a cell stably transfected with both transposons illustrating successful expression of robust voltage-gated sodium current. In this cell, peak sodium current exceeded 5000 pA.

### VI. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Throughout this application, reference is made to various proteins and nucleic acids. It is understood that any names used for proteins or nucleic acids are art-recognized names, such that the reference to the name constitutes a disclosure of the molecule itself.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

By “treating” is meant that an improvement in the disease state, i.e., genetic disorder, autoimmune disease, cancer, viral infection, bacterial infection, or parasitic infection is observed and/or detected upon administration of a substance of the present invention to a subject. Treatment can range from a positive change in a symptom or symptoms of the disease to complete amelioration of the genetic disorder, autoimmune disease, cancer, viral infection, bacterial infection, or parasitic infection, (e.g., reduction in severity or intensity of disease, alteration of clinical parameters indicative of the subject's condition, relief of discomfort or increased or enhanced function), as detected by art-known techniques. The methods of the present invention can be utilized to treat an established genetic disorder, autoimmune disease, cancer, viral infection, bacterial infection, or parasitic infection. One of skill in the art would recognize that genetic disorder, autoimmune disease, cancer, viral infection, bacterial infection, or parasitic infection refer to conditions characterized by the presence of a foreign pathogen or abnormal cell growth. Clinical symptoms will depend on the particular condition and are easily recognizable by those skilled in the art of treating the specific condition.

By “preventing” is meant that after administration of a substance of the present invention to a subject, the subject does not develop the full symptoms of the condition (e.g., genetic disorder, autoimmune disease, cancer, viral, bacterial, or parasitic infection, and/or does not develop the genetic disorder, autoimmune disease, cancer, viral, bacterial, or parasitic infection). Thus, the condition is completely prevented or some recognized symptom or indicia of the condition is prevented or its full manifestation prevented.

By “transposable elements” or “transposon” is meant any genetic construct including but not limited to any gene, gene fragment, or nucleic acid that can be integrated into a target DNA sequence under control of an integrating enzyme.

By “terminal repeat” is meant any repetitive sequence within a sequence of nucleic acids including but not limited to inverted repeats and direct repeats.

By “vector” is meant any composition capable of delivering a nucleic acid, peptide, polypeptide, or protein into a target nucleic acid, cell, tissue, or organism including but not limited to plasmid, phage, transposons, retrotransposons, viral vector, and retroviral vector. “Vector” is also used to refer to a circular or linear polymer of double stranded nucleic acids that is transfected or transformed into a cell.

As used herein, “plasmids” are agents that transport the disclosed nucleic acids into the cell without degradation and allow promoter-driven expression of the protein-encoding nucleic acids (e.g., transgene and integrating enzyme) in the cells into which they are delivered.

By “non-viral vector” is meant any vector that does not comprise a virus or retrovirus.

By “transfection” is meant any method to put nucleic acid into a eukaryotic cell.

By “transformation” is meant any method to put nucleic acid into a prokaryotic cell.

By “cell” is meant any eukaryotic or prokaryotic cell.

By “transgene” is meant any nucleic acid that encodes a gene that is transfected or transformed into a cell.

By “transcriptional unit” is meant a region of DNA that can be transcribed that can be operably linked to a promoter in the vector or put into functional proximity with a promoter upon integration in the genome. In some cases, where the promoter and region of DNA to be transcribed are together in the transcriptional unit, the unit may be referred to as a “cassette,” for example the kanamycin/neomycin resistance cassette. The transcriptional unit can contain regions of DNA that are transcribed to produce mRNAs or regulatory RNAs, with or without promoter sequences.

By “regulatory RNA” is meant, but is not limited to antisense RNAs, small interfering RNAs (siRNA), microRNAs (miRNA), aptamers or ribozymes.

### B. COMPOSITIONS

The invention provides compositions of the invention, comprising a vector containing a transcriptional unit or region to receive a transcriptional unit and an origin of replication functional in a target host cell flanked by minimal piggyBac inverted repeat elements.

Also disclosed are compositions of the invention, wherein the minimal piggyBac inverted repeat (IR) elements are 311 and 236 nucleotides in length. The sequences of the minimal IRs are shown in the Sequence Listing as SEQ ID NO:4, and SEQ ID NO:5.

Also disclosed are compositions of the invention wherein the transcriptional unit is a gene coding sequence with or without a promoter.

Also disclosed are compositions of the invention where the transcriptional unit is an exon with splice donor and splice acceptor cites. For example, disclosed are compositions of the invention where the transcriptional unit contains an exon that is transcribed with mRNA of the gene in which the synthetic transposase has integrated. This would be used in an exon trapping context.

Also disclosed are compositions of the invention, wherein the transcriptional unit is a region of DNA that is transcribed to produce a regulatory RNA.

Also disclosed are compositions of the invention, wherein the gene coding sequence is a selectable marker coding sequence that encodes a polypeptide conferring antibiotic resistance linked to a promoter functional in a target organism, and said antibiotic being selected is from the group consisting of actinomycin, ampicillin, chloramphenicol, erythromycin, gentamycin sulfate, hygromycin, kanamycin, neomycin, penicillin, polymixin B sulfate and streptomycin sulfate.

Also disclosed are compositions of the invention, wherein the gene coding sequence encodes a visible marker. For example the marker can be, but is not limited to the green fluorescent protein (GFP), luciferase, rhodamine, red fluorescent protein, any chimeric protein that includes a light emitting protein, including but not limited to a cyan fluorescent protein-calmodulin chimera or epidermal growth factor receptor GFP-chimera, and any surface protein that could be detected by antibody binding and fluorescent activated cell sorting, including but not limited to CD8 antigen, CD4 antigen, CD22 antigen, or other antigens disclosed herein.

Also disclosed are compositions of the invention, wherein the gene coding sequence is a gene that expresses biological activity. The biological activity can be therapeutic or non-therapeutic as described herein.

The region to receive a gene coding sequence can be a region of nucleotides comprising one or more cloning sites.

Also disclosed are compositions of the invention, wherein the vector contains the piggyBac transposase under the control of a cytomegalovirus (CMV) promoter with an intron between the piggyBac transposase and the CMV promoter.

Also disclosed are compositions of the invention, wherein the intron between the promoter and the transposase is an intron from the virus SV40.

Also disclosed are compositions of the invention, wherein the vector contains in order: a CMV promoter, an intron, a piggyBac transposase coding sequence, a polyadenylation signal, a first minimal piggyBac inverted repeat element, a gene coding sequence or region to receive a gene coding sequence, an origin of replication functional in a target host cell, and a second minimal piggyBac inverted repeat element. In one aspect, the vector is circular. In another aspect the vector is linear. Where the vector is circular, the components listed are present in the order mentioned, beginning with the CMV promoter. Where the vector is linear, the components listed are ordered 5′ to 3 beginning with the CMV promoter.

Also disclosed are compositions of the invention, wherein the piggyBac inverted repeat elements in the helper independent vector (e.g., pPB-Nori) are 311 and 236 nucleotides in length.

A nucleic acid comprising multiple transcriptional units or a combination of transcriptional units and regions to receive a transcriptional unit or multiple regions to receive a transcriptional unit and that are together flanked by piggyBac inverted repeats, wherein each transcriptional unit or region to receive a transcriptional unit is separated from every other transcriptional unit or region to receive a transcriptional unit by an internal ribosome entry site, and operably linked to a promoter such that all transcriptional units are expressed via a bicistronic mRNA.

Also disclosed are compositions of the invention, wherein the vector contains a transgene (gene coding sequence) followed by a selectable marker coding sequence, together flanked by piggyBac inverted repeats and operably linked to a promoter such that transgene and marker coding sequence are expressed via a bicistronic mRNA.

Also disclosed is a vector comprising a first minimal piggyBac inverted repeat element, a promoter (constitutive or inducible), a gene coding sequence or region to receive a gene coding sequence, a IRES element, a second gene coding sequence or region to receive a gene coding sequence, and a second minimal piggyBac inverted repeat element. The insertion of an IRES element allows for a vector with two, three, four, five, etc. transcriptional units or regions to receive a transcriptional units.

Also disclosed is a nucleic acid comprising in 5′ to 3′ order: a first minimal piggyBac inverted repeat element, a promoter (constitutive or inducible), a gene coding sequence (transcriptional unit) or region to receive a gene coding sequence (transcriptional unit), a IRES element, a second gene coding sequence or region to receive a gene coding sequence, a second promoter (constitutive or inducible), a separate and third gene sequence, and a second minimal piggyBac inverted repeat element.

Further disclosed is a a nucleic acid comprising in 5′ to 3′ order: a first minimal piggyBac inverted repeat element, a promoter (constitutive or inducible), a gene coding sequence (transcriptional unit) or region to receive a gene coding sequence (transcriptional unit), a second promoter (same or different, constitutive or inducible), a second gene coding sequence or region to receive a gene coding sequence, and a second minimal piggyBac inverted repeat element.

Also disclosed are compositions of the invention, wherein the vector contains a chimeric zinc finger piggyBac transposase.

Also disclosed are compositions of the invention, wherein the vector contains a humanized piggyBac transposase. For example, see SEQ ID NO:14.

Also disclosed are compositions of the invention, wherein the nucleic acid is present in a non-viral vector.

In some embodiments the promoter and/or enhancer is derived from either a virus or a retrovirus. Also disclosed are compositions of the invention, wherein the promoter element is a promoter/enhancer.

Also disclosed are compositions of the invention, wherein the promoter is a site-specific promoter.

It has been shown that all specific regulatory elements can be cloned and used to construct expression vectors that are selectively expressed in specific cell types. The site-specific promoter can be selected at least from the group consisting of the glial fibrillary acetic protein (GFAP) promoter, myelin basic promoter (MBP), MCK promoter, NSE promoter, nestin promoter, nestin promoter, synapsin promoter, Insulin 2 (Ins2) promoter, PSA promoter, albumin promoter, TRP-1 promoter and the tyrosinase promoter. Also disclosed is a promoter specific for breast tissue, such as the WAP promoter, a promoter specific for ovarian tissue, such as the ACTB promoter, or a promoter specific for bone tissue. Any tissues specific promoter can be used.

Also disclosed are compositions of the invention, wherein the promoter is inducible. The inducible promoter can be selected at least from the group consisting of human heat shock promoter, Egr-1 promoter, tetracycline promoter, and the human glandular kallikrien 2 (hK2) promoter.

As the transposable element will need to be integrated into the host genome, an intergrating enzyme is needed. Intergrating enzymes can be any enzyme with integrating capabilities. Such enzymes are well known in the art and can include but are not limited to transposases, integrases (including DDE transposases), recombinases including but not limited to tyrosine site-specific recombinases (integrase) and other site-specific recombinases (e.g., cre), bacteriophage integrases, retrotransposases, and retroviral integrases. Thus, provided is a composition wherein the integrating enzyme is the piggyBac transposase.

The integrating enzyme can be a chimeric integrating enzyme. The chimeric integrating enzymes of the present invention comprise two components: DNA docking factor (first domain) (e.g., DNA Binding Domain (DBD)) and an integrating (enzymatic) domain (second domain). The DNA docking factor can be arranged anywhere in relation to the integrating domain (e.g. internally, or at the amino or carboxyl termini). Furthermore, a portion of the wild-type integrating enzyme, for example, the portion that has the DBD of the native enzyme, could be deleted and replaced with a DBD that recognizes DNA of the target cell. The chimeric proteins of the invention comprise a first domain that attaches the chimeric protein to target nucleic acid, and a second domain that integrates donor nucleic acid (transgene) into the target nucleic acid. As employed herein, the phrase “chimeric protein” refers to a genetically engineered recombinant protein wherein the domains thereof are derived from heterologous coding regions (i.e., coding regions obtained from different genes). General molecular methods, and specifically those of Katz et al. (U.S. Pat. No. 6,150,511, incorporated herein by reference) can be used to construct a chimeric transposase of the invention. Provided is a chimeric piggyBac transposase in accordance with the teaching herein.

The chimeric integrating enzyme proteins of the invention are prepared by recombinant DNA methods, in which the DNA sequences encoding each domain are “operably linked” together such that upon expression, a fusion protein is generated having the targeting and transposase functions described previously. As used herein, the term “operably linked” means that the DNA segments encoding the fusion protein are assembled with respect to each other, and with respect to an expression vector in which they are inserted, in such a manner that a functional fusion protein is effectively expressed.

As used herein, “first domain” refers to the domain within the chimeric protein that functions to attach the chimeric protein to a specific recognition sequence on a target nucleic acid. The first domain is at least 5 amino acids in length and can be located anywhere within the chimeric protein, e.g., internally, or at the amino or carboxyl termini thereof. The first domain can be a DNA docking factor, either a “DNA-binding domain” or a “protein-binding domain” that is operative to couple and/or associate the chimeric protein with a recognition sequence on the target nucleic acid.

By “DNA docking factor” is meant any amino acid sequence that associates with DNA directly or indirectly. Thus when the association of the chimeric integrating enzyme with the target nucleic acid occurs by indirect binding, a protein-binding domain is employed as the docking factor. Suitable protein-binding domains can be obtained from viral transcription factors (e.g., HSV-VP16 and adenovirus E1A) and cellular transcription factors. Throughout the present disclosure, the terms DNA binding domain, DNA directing factor, and protein binding domain are used to refer to DNA docking factors. It is understood that these terms may be used interchangeably throughout the present invention without affecting the overall goal of the invention.

As used herein, the term “DNA-binding domain” encompasses a minimal peptide sequence of a DNA-binding protein, up to the entire length of a DNA-binding protein without losing function. When a DNA-binding domain is employed in the invention, the association of the chimeric integrating enzyme with the target nucleic acid occurs by direct interaction with the host nucleic acid. The DNA-binding domain brings the second domain (i.e., the integrating domain) in close proximity to a specific recognition sequence on the target nucleic acid so that a desired donor nucleic acid can be integrated into the target nucleic acid sequence.

DNA-binding domains are typically derived from DNA-binding proteins. Such DNA-binding domains are known to function heterologously in combination with other functional protein domains by maintaining the ability to bind the natural DNA recognition sequence (see, e.g., Brent and Ptashne, 1985, Cell, 43:729-736 incorporated herein by reference in its entirety). For example, hormone receptors are known to have interchangeable DNA-binding domains that function in chimeric proteins (see, e.g., U.S. Pat. No. 4,981,784; and Evans, R., 1988, Science, 240:889-895 incorporated by reference herein in its entirety).

“DNA-binding protein(s)” utilized herein belong to a well-known class of proteins that are able to directly bind DNA and perform a variety of functions, such as facilitate initiation of transcription or repression of transcription. Exemplary DNA-binding proteins for use herein include transcription control proteins (e.g., transcription factors and the like; Conaway and Conaway, 1994, “Transcription Mechanisms and Regulation”, Raven Press Series on Molecular and Cellular Biology, Vol. 3, Raven Press, Ltd., New York, N.Y.; incorporated herein by reference in its entirety); recombination enzymes (e.g., hin recombinase, and the like); and DNA modifying enzymes (e.g., restriction enzymes, and the like).

Transcription factors with DNA-binding proteins suitable for use herein include, e.g., homeobox proteins, zinc finger proteins, hormone receptors, helix-turn-helix proteins, helix-loop-helix proteins, basic-Zip proteins (bZip), beta-ribbon factors, and the like. See, for example, Harrison, S., “A Structural Taxonomy of DNA-binding Domains,” Nature, 353:715-719.

Homeobox DNA-binding proteins suitable for use herein include, but are not limited to HOX, STF-1 (Leonard et al., 1993, Mol. Endo., 7:1275-1283), Antp, Mat, alpha.-2, INV, and are incorporated by reference herein in their entirety (see, also, Scott et al. (1989), Biochem. Biophys. Acta, 989:25-48). It has been found by Leonart et al., that a fragment of 76 amino acids (corresponding to a.a. 140-215 described in Leonard et al., 1993, Mol. Endo., 7:1275-1283) containing the STF-1 homeodomain binds DNA as tightly as wild-type STF-1 and is incorporated by reference herein in its entirety.

Zinc fingers can be manipulated to recognize a broad range of sequences. As such, these enzymes can direct cleavage to arbitrarily chosen targets. A double-strand break (DSB) in the chromosomal target greatly enhances the frequency of localized recombination events. Zinc-finger nucleases (ZFNs) have a DNA recognition domain composed of three Cys₂His₂ zinc fingers linked to a nonspecific DNA cleavage domain (Y. G. Kim et al. (1996) Proc. Natl. Acad. Proc. Natl. Acad. Sci. U.S.A. 93, 1156). To act as a nuclease, the cleavage domain can dimerize (J. Smith et al. (2000) Nucleic Acids Res. 28, 3361). This can be achieved by providing binding sites for two sets of zinc fingers in close proximity and in the appropriate orientations (J. Smith et al. (2000) Nucleic Acids Res. 28, 3361; M. Bibikova et al. (2001) Mol. Cell. Biol. 21, 289). Suitable zinc finger DNA-binding proteins provided for use herein include but are not limited to Zif268, GLI, and XFin. These proteins can be found throughout the literature via Klug and Rhodes (1987), Trends Biochem. Sci., 12:464; Jacobs and Michaels (1990), New Biol., 2:583; and Jacobs (1992), EMBO J., 11:4507-4517 (incorporated by reference herein in their entirety). Thus, provided is a composition comprising a zinc finger coding sequence linked to piggyBac transposase.

Exemplary hormone receptor DNA-binding proteins for use herein include but are not limited to glucocorticoid receptor, thyroid hormone receptor, and estrogen receptor are described in the literature (U.S. Pat. Nos. 4,981,784; 5,171,671; and 5,071,773, incorporated by reference herein in their entirety).

Suitable helix-turn-helix DNA-binding proteins for use herein include but are not limited to lambda-repressor, cro-repressor, 434 repressor, and 434-cro. These helix-turn-helix DNA-binding proteins are provided (Pabo and Sauer, 1984, Annu. Rev. Biochem., 53:293-321 incorporated herein by reference in their entirety).

Exemplary helix-loop-helix DNA-binding proteins for use herein include but are not limited to MRF4 (Block et al., 1992, Mol. and Cell Biol., 12(6): 2484-2492, incorporated herein by reference), CTF4 (Tsay et al., 1992, NAR, 20(10): 2624, incorporated herein by reference), NSCL, PAL2, and USF. See, for review, Wright (1992), Current Opinion in Genetics and Development, 2(2):243-248; Kadesch, T. (1992), Immun. Today, 13(1): 31-36; and Garell and Campuzano (1991), Bioessays, 13(10): 493-498, which are incorporated herein by reference.

Exemplary basic Zip DNA-binding proteins for use herein include but are not limited to GCN4, fos, and jun (see, for review, Lamb and McKnight, 1991, Trends Biochem. Sci., 16:417-422 incorporated herein by reference). Exemplary beta.-ribbon factors provided for use herein include, Met-J, ARC, and MNT.

Recombination enzymes with suitable DNA-binding proteins for use herein include but are not limited to the hin family of recombinases (e.g., hin, gin, pin, and cin; see, Feng et al., Feng et al., 1994, Science, 263:348-355, incorporated herein by reference), the lambda.-integrase family, flp-recombinase, TN916 transposons, and the resolvase family (e.g., TN21 resolvase).

DNA-modifying enzymes with suitable DNA-binding proteins for use herein include, for example, restriction enzymes, DNA-repair enzymes, and site-specific methylases. For use in the instant invention, restriction enzymes are modified using methods well-known in the art to remove the restriction digest function from the protein while maintaining the DNA-binding function (see, e.g., King et al., 1989, J. Biol. Chem., 264 (20):11807-11815, incorporated herein by reference). Thus, any restriction enzyme can be employed herein. The utilization of a restriction enzyme recognizing a rare DNA sequence permits attachment of the invention chimeric protein to relatively few sites on a particular stretch of genomic DNA.

The modification of existing DNA-binding domains to recognize new target recognition sequences is also contemplated herein. It has been found that in vitro evolution methods can be applied to modify and improve existing DNA-binding domains. Devlin et al., 1990, Science, 249:404-406; and Scott and Smith, 1990, Science, 249:386-390 are incorporated herein by reference in their entirety for teachings on modification of existing DNA-binding domains.

“Protein-binding domain(s)” suitable for use as the “first domain” of the invention chimeric protein is typically derived from proteins able to bind another protein (e.g., a transcription factor) that is either directly or indirectly attached (coupled) to the target nucleic acid sequence. Thus, when a protein-binding domain is employed as the first domain, the association of the invention chimeric protein with the target nucleic acid occurs by indirect binding. Suitable protein-binding domains can be obtained, for example, from viral transcription factors (e.g., HSV-VP16, adenovirus E1A, and the like), cellular transcription factors, and the like using routine molecular methods.

In addition to readily available protein-binding domains, small protein-binding domains, e.g., in the range of about 5-25 amino acids, can be obtained employing “phage display library” methods described (Rebar and Pabo, 1994, Science, 263:671-673). It has been found that short peptides can be isolated using phage display libraries that bind to a selected protein. For example, a peptide was obtained from a library displaying random amino-acid hexamers on the surface of a phage that bound specifically to avidin; this peptide bore no similarity to any known avidin ligands (Devlin et al., 1990, Science, 249:404-406). This well-known method is used to This well-known method is used to create protein-binding domains that bind to proteins already bound in vivo to desired target nucleic acid.

Microsatellite regions are repetitive sequences in the genome. By targeting repetitive sequences whether through a chimeric integrating enzyme or through homologous sequences one can target integration into non-transcribed regions of the genome (i.e. eliminating the risk of insertional mutagenesis) and by having more targets increasing the efficiency of integration, i.e. many targets are better than one. There are repetitive, non-coding regions in the genome that allow integration as described herein, followed by transcription of the transgene driven by the promoter provided in the construct.

The chimeric integrating enzyme of the invention comprises an integrating (enzymatic) domain (second domain). The integrating domain comprises or is derived from an integrating enzyme. Intergrating enzymes can be any enzyme with integrating capabilities. Such enzymes are well known in the art and can include but are not limited to transposases, integrases (including DDE transposases), tyrosine site-specific recombinases (integrase), recombinases, site-specific recombinases (e.g., cre), bacteriophage integrases, integron, retrotransposases, retroviral integrases and terminases.

Disclosed are compositions, wherein the integrating enzyme is a transposase. It is understood and herein contemplated that the transposase of the composition is not limited and to any one transposase and can be selected from at least the group consisting of piggyBac, Sleeping Beauty (SB), Tn7, Tn5, mos1, Himar1, Hermes, Tol2 element, Pokey, Minos, S elements, P-element, ICESt1, Quetzal elements, Tn916, maT, Tc1/mariner and Tc3.

Where the integrating enzyme is a transposase, it is understood that the transposase of the composition is not limited and to any one transposase and can be selected from at least the group consisting of piggyBac, Sleeping Beauty (SB), Tn7, Tn5, Tn916, Tc1/mariner, Minos and S elements, Quetzal elements, Txr elements, maT, mos1, Himar1, Hermes, Tol2 element, Pokey, P-element, and Tc3. Additional transposases can be found throughout the art, for example, U.S. Pat. No. 6,225,121, U.S. Pat. No. 6,218,185 U.S. Pat. No. 5,792,924 U.S. Pat. No. 5,719,055, U.S. Patent Application No. 20020028513, and U.S. Patent Application No. 20020016975 and are herein incorporated by reference in their entirety. Since the applicable principal of the invention remains the same, the compositions of the invention can include chimeric transposases constructed from transposases not yet identified.

Also disclosed are integrating enzymes of the disclosed compositions wherein the enzyme is an integrase. For example, the integrating enzyme can be a bacteriophage integrase. Such integrase can include any bacteriophage integrase and can include but is not limited to lamda (λ) bacteriophage and mu (μ) bacteriophage, as well as Hong Kong 022 (Cheng Q., et al. Specificity determinants for bacteriophage Hong Kong 022 integrase: analysis of mutants with relaxed core-binding specificities. (2000) Mol Microbiol. 36(2):424-36.), HP1 (Hickman, A. B., et al. (1997). Molecular organization in site specific recombination: The catalytic domain of bacteriophage HP1 integrase at 2.7 A resolution. Cell 89: 227-237), P4 (Shoemaker, N B, et al. (1996). The Bacteroides mobilizable insertion element, NBU1, integrates into the 3′ end of a Leu-tRNA gene and has an integrase that is a member of the lambda integrase family. J Bacteriol. 178(12):3594-600.), P1 (Li Y, and Austin S. (2002) The P1 plasmid in action: time-lapse photomicroscopy reveals some unexpected aspects of plasmid partition. Plasmid. 48(3):174-8.), and T7 (Rezende, L. F., et al. (2002) Essential Amino Acid Residues in the Single-stranded DNA-binding Protein of Bacteriophage T7. Identification of the Dimer Interface. J. Biol. Chem. 277, 50643-50653.).

Integrase maintains its activity when fused to other proteins. This has been demonstrated by the use of the lambda repressor-integrase (40) and maltose binding protein-integrase fusion proteins (41). Additionally, chimeric recombinases, transcription factors, oncogenes, etc. have maintained their activity when fused to other protein domains (42). However, attempts of in vivo targeting of site-selective retroviruses that included sequences encoding integrase fusion proteins have not yet been demonstrated (43-45). The Tc1/mariner elements are promiscuous and have been successfully used as transgene vectors from one species to another in flies (49-53), mosquitoes (54), bacteria (55), protozoa (56), and vertebrates.

Also disclosed are integrating enzymes of the disclosed compositions wherein the enzyme is a recombinase. For example, the recombinase can be a Cre recombinase, Flp recombinase, HIN recombinase, or any other recombinase. Recombinases are well-known in the art. An extensive list of recombinases can be found in Nunes-Duby S E, et al. (1998) Nuc. Acids Res. 26(2): 391-406, which is incorporated herein in its entirety for its teachings on recombinases and their sequences.

Also disclosed are integrating enzymes of the disclosed compositions wherein the enzyme is a retrotransposase. For example, the retrotransposase can be a Gate retrotransposase (Kogan G L, et al. (2003) The GATE retrotransposon in Drosophila melanogaster: mobility in melanogaster: mobility in heterochromatin and aspects of its expression in germline tissues. Mol Genet Genomics. 269(2):234-42).

The chimeric integrating enzyme of the invention can have the host specific binding domain fused to the transposase's N-terminus.

The chimeric integrating enzyme of the invention can have the host specific binding domain is fused to the transposase's C-terminus.

Also provided are compositions comprising a nucleic acid for a transgene under the control of a promoter element flanked by two internal repeats and a nucleic acid enocoding a integrating enzyme under the control of a promoter element. Some internal repeats (e.g., some short and long interspersed nuclear elements), incorporated herein by reference to the art that discloses them, are permissive for site-selective integration (68-69) and would allow for transgene expression even without nuclear matrix attachment regions flanking the transgene (66-67). Proteins that selectively bind to interspersed repeat elements have been identified (70-73) and are herein incorporated by reference. Development of fusion proteins incorporating DNA binding domains to known transcription-permissive, repetitive DNA sequences allow targeted integration as described earlier.

Also provided is a nucleic acid in which the transgene is flanked by the inverted terminal repeats. In this embodiment, the terminal repeats can be derived from known transposons. Examples of transposons include, but are not limited to the following: piggyBac (Tamura T, et al. Germline transformation of the silkworm Bombyx mori L. using a piggyBac transposon-derived vector. Nat Biotechnol. 2000 January; 18(1):81-4), Sleeping Beauty (Izsvak Z, Ivics Z, and Plasterk R H. (2000) Sleeping Beauty, a wide host-range transposon vector for genetic transformation in vertebrates. J. Mol. Biol. 302:93-102), mos1 (Bessereau J L, et al. (2001) Mobilization of a Drosophila transposon in the Caenorhabditis elegans germ line. Nature. 413(6851):70-4; Zhang L, et al. (2001) DNA-binding activity and subunit interaction of the mariner transposase. Nucleic Acids Res. 29(17):3566-75, Himar1 (Lampe D J, et al. (1998) Factors affecting transposition of the Himar1 mariner transposon in vitro. Genetics. 149(1): 179-87), Hermes, Tol2 element, Pokey, Tn5 (Bhasin A, et al. (2000) Characterization of a Tn5 pre-cleavage synaptic complex. J Mol Biol 302:49-63), Tn7 (Kuduvalli P N, Rao J E, Craig N L. (2001) Target DNA structure plays a critical role in Tn7 transposition. EMBO J 20:924-932), Tn916 (Marra D, Scott J R. (1999) Regulation of excision of the conjugative tranposon Tn916. Mol Microbiol 2:609-621), Tc1/mariner (Izsvak Z, Ivics Z, Hackett P B. (1995) Characterization of a Tc-1 like transposable element in zebrafish (Danio rerio). Mol. Gen. Genet. 247:312-322), transposable element in zebrafish (Danio rerio). Mol. Gen. Genet. 247:312-322), Minos and S elements (Franz G and Savakis C. (1991) Minos, a new transposable element from Drosophila hydei, is a member of the Tc1-like family of transposons. Nucl. Acids Res. 19:6646; Merriman P J, Grimes C D, Ambroziak J, Hackett D A, Skinner P, and Simmons M J. (1995) S elements: a family of Tc1-like transposons in the genome of Drosophila melanogaster. Genetics 141:1425-1438), Quetzal elements (Ke Z, Grossman G L, Cornel A J, Collins F H. (1996) Quetzal: a transposon of the Tc1 family in the mosquito Anopheles albimanus. Genetica 98:141-147); Txr elements (Lam W L, Seo P, Robison K, Virk S, and Gilbert W. (1996) Discovery of amphibian Tc1-like transposon families. J Mol Biol 257:359-366), Tc1-like transposon subfamilies (Ivics Z, Izsvak Z, Minter A, Hackett P B. (1996) Identification of functional domains and evolution of Tc1-like transposable elements. Proc. Natl. Acad Sci USA 93: 5008-5013), Tc3 (Tu Z, Shao H. (2002) Intra- and inter-specific diversity of Tc-3 like tranposons in nematodes and insects and implications for their evolution and transposition. Gene 282:133-142), ICESt1 (Burrus V et al. (2002) The ICESt1 element of Streptococcus thermophilus belongs to a large family of integrative and conjugative elements that exchange modules and change their specificity of integration. Plasmid. 48(2): 77-97), maT, and P-element (Rubin G M and Spradling A C. (1983) Vectors for P element mediated gene transfer in Drosophila. Nucleic Acids Res. 11:6341-6351). These references are incorporated herein by reference in their entirety for their teaching of the sequences and uses of transposons and transposon ITRs. In one aspect, the terminal repeats can be minimal inverted repeats, for example, those disclosed in SEQ ID NOS: 4 and 5. These IRs contain binding sites for the piggyBac transposase.

Translocation of Sleeping Beauty (SB) transposon requires specific binding of SB transposase to inverted terminal repeats (ITRs) of about 230 bp at each end of the transposon, which is followed by a cut-and-paste transfer of the transposon into a target DNA sequence. The ITRs contain two imperfect direct repeats (DRs) of about 32 bp. The outer DRs are at the extreme ends of the transposon whereas the inner DRs are located inside the transposon, 165-166 bp from the outer DRs. Cui et al. (J. Mol Biol 318:1221-1235) investigated the roles of the DR elements in transposition. Within the 1286-bp element, the essential regions are contained in the intervals bounded by coordinates 229-586, 735-765, and 939-1066, numbering in base pairs from the extreme 5′ end of the element. These regions may contain sequences that are necessary for transposase binding or that are needed to maintain proper spacing between binding sites.

Transposons are bracketed by terminal inverted repeats that contain binding sites for the transposase. Elements of the IR/DR subgroup of the Tc1/mariner superfamily have a pair of transposase-binding sites at the ends of the 200-250 bp long inverted repeats (IRs) (Izsvak, et al. 1995). The binding sites contain short, 15-20 bp direct repeats (DRs). This characteristic structure can be found in several elements from evolutionarily distant species, such as Minos and S elements in flies (Franz and Savakis, 1991; Merriman et al, 1995), Quetzal elements in mosquitos (Ke et al, 1996), Txr elements in frogs (Lam et al, 1996) and at least three Tc1-like transposon subfamilies in fish (Ivics et al., 1996), including SB [Sleeping Beauty] and are herein incorporated by reference.

Whereas Tc1 transposons require one binding site for their transposase in each IR, Sleeping Beauty requires two direct repeat (DR) binding sites within each IR, and is therefore classified with Tc3 in an IR/DR subgroup of the Tc1/mariner superfamily (96,97). Sleeping Beauty transposes into TA dinucleotide sites and leaves the Tc1/mariner characteristic footprint, i.e., duplication of the TA, upon excision. The non-viral plasmid vector contains the transgene that is flanked by IR/DR sequences, which act as the binding sites for the transposase. The catalytically active tranposase can be expressed from a separate (trans) or same (cis) plasmid system. The transposase binds to the IR/DRs, catalyzes the excision of the flanked transgene, and mediates its integration into the target host genome.

Tc3 of Caenorhabditis elegans is one of the founding members of the Tc1 family which includes DNA transposons in vertebrates, insects, nematodes and fungi. Tu A, et al. (Gene 282:133-142) present the characterization of a number of Tc3-like transposons in C. elegans, Caenorhabditis briggsae, and Drosophila melanogaster, which has revealed high levels of inter- and intra-specific diversity and further suggests a broad distribution of the Tc3-like transposons. These newly defined transposons and the previously described Tc3 and MsqTc3 form a highly divergent yet distinct clade in the Tc1 family. The majority of the Tc3-like transposons contain two putative binding sites for their transposases. The first is near the terminus and the second is approximately 164-184 bp from the first site. There is a large amount of variation in the length (27-566 bp) and structure of the terminal inverted repeats (TIRs) of Tc3-like transposons.

Mos1 is a member of the mariner/Tc1 family of transposable elements originally identified in Drosophila mauritiana. It has 28 bp terminal inverted repeats and like other elements of this type it transposes by a cut and paste mechanism, inserts at TA dinucleotides and codes for a transposase. This is the only protein required for transposition in vitro. Zhang and in vitro. Zhang and colleagues (Nucleic Acids Res 29:3566-3575) have investigated the DNA binding properties of Mos1 transposase and the role of transposase-transposase interactions in transposition. Purified transposase recognises the terminal inverted repeats of Mos1 due to a DNA-binding domain in the N-terminal 120 amino acids. This requires a putative helix-turn-helix motif between residues 88 and 108. Binding is preferentially to the right hand end, which differs at four positions from the repeat at the left end. Cleavage of Mos1 by transposase is also preferentially at the right hand end.

Based upon the requirements for integration of the transposable elements, it appears a host DNA directing factor is necessary for efficient integration by juxtaposing the transposon-transposase complex adjacent to the host DNA. Indeed, Tc1/mariner transposases do have DNA binding domains. However, these DNA binding domains apparently are not site selective (35), possibly lack strong recognition sites in certain host genomes, and may require other host proteins for efficient integration by docking the transposon-transposase to the host DNA.

The invention overcomes this shortcoming by providing compositions comprising a non-viral vector further comprising a chimeric integrating enzyme (i.e., integrating enzyme-host DNA binding domain) to bypass the potential requirement of a host DNA directing factor(s) for efficient, site-selective integration. It is understood that the chimeric integrating enzyme can include but is not limited to chimeric transposases, chimeric integrases, chimeric retrotransposases, retroviral integrases, integrons, and chimeric recombinases.

Thus, disclosed are compositions comprising a transgene flanked by terminal repeats of a transposable element, e.g. piggyBac, and a required chimeric enzyme (e.g., host DNA binding domain-transposase) in a non-viral packaging system for targeted integration into the host genome. In one aspect, this chimeric enzyme that is site-selective would substitute the native DNA binding domain of the integrating enzyme with one that is host specific and site-selective, thereby bypassing the requirement of a host-DNA directing factor. In a further aspect, the native piggyBac transposase is intact, but a zinc finger DNA binding domain is added to the N-terminus to facilitate target-specific integration.

Also disclosed are compositions of the invention, wherein the transposase is a chimeric transposase comprising a host-specific or site-specific DNA binding domain.

Thus, disclosed are chimeric transposases and the transposons that are used to introduce nucleic acid sequences into the DNA of a cell. A transposase is an enzyme that is capable of binding to DNA at regions of DNA termed inverted repeats. Transposons typically contain at least one, and preferably two, inverted repeats that flank an intervening nucleic acid sequence. The transposase binds to recognition sites in the inverted repeats and catalyzes the incorporation of the transposon into host DNA. Transposon function is frequently limited to the host species. Even in those transposons that are not limited to their “normal host” the efficiency of integration varies dramatically. This invention can increase the efficiency of integration by modifying a transposase to include a host DNAbinding domain (whether for the purpose of site selectiveness or not) as described herein. The novel DNA binding domain of this chimeric transposase can be added to the native transposases or it can substitute for the DNA binding domain of the native transposase. Thus, the host DNA [directing factor] chimeric transposase, recognition sites on the plasmid that would recognize an endogenous protein (or a newly introduced protein) that would then direct the complex to the vicinity of the host-DNA, incorporating host-like sequences (e.g., repetitive sequences) or a combination of the above play roles in the site-selective and/or efficient transgene integration provided by the present invention.

Gene transfer vectors for gene therapy can be broadly classified as viral vectors or non-viral vectors. The use of the disclosed vectors provides an important and suprising improvement over the non-viral DNA-mediated gene transfer. Up to the present time, viral vectors have been the focus of gene therapy efforts, because they have been found to be more efficient at introducing and expressing genes in cells than non-viral vectors. Once the efficiency problems of the prior art are overcome, as taught herein, there are several advantages to non-viral gene transfer over virus-mediated gene transfer for the development of new gene therapies. For example, adapting viruses as agents for gene therapy restricts genetic design to the constraints of that virus genome in terms of size, structure and regulation of expression. Non-viral vectors are generated largely from synthetic starting materials and are therefore more easily manufactured than viral vectors. Non-viral reagents are less likely to be immunogenic than viral agents making repeat administration possible. Non-viral vectors are more stable than viral vectors and therefore are better suited for pharmaceutical formulation and application than are viral vectors.

In past embodiments, non-viral gene transfer systems have not been equipped to promote integration of nucleic acid into the DNA of a cell, including host chromosomes. As a result, stable gene transfer frequencies using non-viral systems have been very low; 0.1% at best in tissue culture cells and much less in primary cells and tissues. The prior art efforts at transposon-based non-viral vectors have attempted to provide a non-viral gene transfer system that facilitates integration and markedly improves the frequency of stable gene transfer. However, the integration is not site specific and is not uniformly efficient, and may vary markedly depending upon the host cell line. The disclosed compositions allow for site-selective integration into the host genome, and provide the suprising advantage of efficient integration in those hosts that do not have the required DNA directing factor as mentioned herein.

In the gene transfer system of this invention, the chimeric integrating enzyme can be introduced into the cell as a protein or as nucleic acid encoding the protein. In one embodiment the nucleic acid encoding the protein is RNA and in another, the nucleic acid is DNA. Further, nucleic acid encoding the chimeric transposase protein can be incorporated into a cell through a viral vector, cationic lipid, or other standard transfection mechanisms including electroporation or particle bombardment used for eukaryotic cells. Following or concurrent with introduction of the nucleic acid encoding chimeric transposae, the nucleic acid comprising a transposon can be introduced into the same cell. Alternatively the nucleic acid encoding the chimeric transposase can be the same nucleic acid that includes the trangene and terminal repeats.

Similarly, the nucleic acid can be introduced into the cell as a linear fragment or as a circularized fragment. Preferably the nucleic acid sequence comprises at least a portion of an open reading frame to produce a functional amino-acid containing product. In a preferred embodiment the nucleic acid sequence encodes at least one active or functional peptide, polypeptide, or protein, and includes at least one promoter selected to direct expression of the open reading frame or coding region of the nucleic acid sequence. The protein encoded by the nucleic acid sequence can be any of a variety of recombinant proteins new or known in the art. In one embodiment the protein encoded by the nucleic acid sequence is a marker protein such as green fluorescent protein (GFP), chloramphenicol acetyltransferase (CAT), growth hormones, for example to promote growth in a transgenic animal, beta-galactosidase (lacZ), luciferase (LUC), and insulin-like growth factors (IGFs).

The gene transfer system of this invention can readily be used to produce transgenic animals that carry a particular marker or express a particular protein in one or more cells of the animal. Methods for producing transgenic animals are known in the art and the incorporation of the gene transfer system of this invention into these techniques does not require undue experimentation. Further, a review of the production of biopharmaceutical proteins in the milk of transgenic dairy animals (see Young et al., BIO PHARM (1997), 10, 34-38) and the references provided therein, detail methods and strategies for producing recombinant proteins in milk and are encorporated herein in their entirety for teachings related to production of biopharmaceutical proteins. The methods and the gene transfer system of disclosed herein can be readily incorporated into these transgenic techniques without undue experimentation in view of what is known in the art and particularly in view of this disclosure.

In one embodiment of a transgenic animal, wherein the transgenic animal acts as a bioreactor, the protein is a product for isolation from a cell. Transgenic animals as bioreactors are known. Protein can be produced in quantity in milk, urine, blood or eggs. Promoters are known that promote expression in milk, urine, blood or eggs and these include, but are not limited to, casein promoter, the mouse urinary protein promoter, beta-globin promoter and the ovalbumin promoter respectively. Recombinant growth hormone, recombinant insulin, and a variety of other recombinant proteins have been produced using other methods for producing protein in a cell. Nucleic acids encoding these or other proteins can be incorporated into the nucleic acid fragment of this invention and introduced into a cell. Efficient incorporation of the nucleic acid fragment into the DNA of a cell occurs when a chimeric transposase as described herein is present. Where the cell is part of a tissue or part of a transgenic animal, large amounts of recombinant protein can be obtained. There are a variety of methods for producing transgenic animals for research or for protein production. The following references are incorporated herein in their entirety for their teachings on methods of producing transgenic animals (Hackett et al. (1993). The molecular biology of transgenic fish. In Biochemistry and Molecular Biology of Fishes (Hochachka & Mommsen, eds) Vol. 2, pp. 207-240. Other methods for producing transgenic animals include the teachings of M. Markkula et al., Rev. Reprod., 1, 97-106 (1996); R. T. Wall et al., J. Dairy Sci, 80, 2213-2224 (1997); J. C. Dalton, et al., Adv. Exp. Med. Biol., 411, 419-428 (1997); and H. Lubon et al., Transfus. Med. Rev., 10, 131-143 (1996). Transgenic zebrafish were made, as described by Hackett et al (Patent Application #20020016975). Transposon-based systems have also been tested through the introduction of the nucleic acid systems have also been tested through the introduction of the nucleic acid with a marker protein into mouse embryonic stem cells (ES) and it is known that these cells can be used to produce transgenic mice (A. Bradley et al., Nature, 309, 255-256 (1984)).

In general, there are two methods to achieve improved stocks of commercially important animals. The first is classical breeding, which has worked well for land animals, but it takes decades to make major changes. A review by Hackett et al. (1997) points out that by controlled breeding, growth rates in coho salmon (Oncorhynchus kisutch) increased 60% over four generations and body weights of two strains of channel catfish (Ictalurus punctatus) were increased 21 to 29% over three generations. The second method is genetic engineering, a selective process by which genes are introduced into the chromosomes of animals or plants to give these organisms a new trait or characteristic, like improved growth or greater resistance to disease. The results of genetic engineering have exceeded those of breeding in some cases. In a single generation, increases in body weight of 58% in common carp (Cyprinus carpio) with extra rainbow trout growth hormone I genes, more than 1000% in salmon with extra salmon growth hormone genes, and less in trout were obtained. The advantage of genetic engineering in fish, for example, is that an organism can be altered directly in a very short periods of time if the appropriate gene has been identified (see Hackett, 1997). The disadvantage of genetic engineering in fish is that few of the many genes that are involved in growth and development have been identified and the interactions of their protein products is poorly understood. Procedures for genetic manipulation are lacking many economically important animals. The present invention provides an efficient system for performing insertional mutagenesis (gene tagging) and efficient procedures for producing transgenic animals.

The disclosed transposon-based system has applications to many areas of biotechnology. Development of transposable elements for vectors in animals permits the following: 1) efficient insertion of genetic material into animal chromosomes using the methods given in this application; 2) generation of multi-gene transgenic animals; 3) identification, isolation, and characterization of genes involved with growth and development through the use of transposons as insertional mutagens (e.g., see Kaiser et al., 1995, “Eukaryotic transposable elements as tools to study gene structure and function.” In Mobile Genetic Elements, IRL Press, pp. 69-100) which is incorporated herein by reference in its entirety; 3) identification, isolation and characterization of transcriptional regulatory sequences controlling growth and development; 4) use of marker constructs for quantitative trait loci (QTL) analysis; and 5) identification of trait loci (QTL) analysis; and 5) identification of genetic loci of economically important traits, besides those for growth and development, i.e., disease resistance (e.g., Anderson et al., 1996, Mol. Mar. Biol. Biotech., 5, 105-113) which is incorporated herein by reference in its entirety. In one example, the system of this invention can be used to produce sterile transgenic fish. Broodstock with inactivated genes could be mated to produce sterile offspring for either biological containment or for maximizing growth rates in aquacultured fish. Thus, provided are transgenic animals, generated by the use of the present vectors and method, including transgenic rodents (e.g., rat, mice, guinea pig, etc.), transgenic fish (e.g., zebrafish, trout, salmon, catfish, etc.), transgenic livestock (e.g., cattle, horses, sheep, pigs, etc.), transgenic c-elegans, insects (e.g., mosquitos, beetles, etc.). In one aspect, the transgenic insect is not a mosquito.

The compositions and methods of the present invention are also useful for the introduction of a nucleic acid sequence of interest into a plant cells to produce transgenic plants. As used herein, the term “transgenic plant” refers to the introduction of foreign nucleic acid sequences into the nuclear, mitochondrial or plastid genome of a plant. As used herein, the term “plant” is defined as a unicellular or multicellular organism capable of photosynthesis. This includes the prokaryotic and eukaryotic algae (including cyanophyta and blue-green algae), eukaryotic photosynthetic protists, non-vascular and vascular multicellular photosynthetic organisms, including angiosperms (monocots and dicots), gymnosperms, spore-bearing and vegetatively-reproducing plants. Also included are unicellular and multicellular fungi.

Production of a transgenic plant can be accomplished by modifying an isolated transposable element of the type described herein to include the nucleic acid sequence of interest flanked by the termini of the isolated transposable element. The modified transposable element can be introduced into a plant cell in the presence of a transposase protein or a nucleic acid sequence encoding a transposase or a virus encoding a transposase protein (e.g., helper plasmid) using techniques well known in the art. Exemplary techniques are discussed in detail in Gelvin et al., “Plant Molecular Biology Manual”, 2nd Ed., Kluwen Academic Publishers, Boston (1995), the teachings of which are incorporated herein by reference. The transposase (along with DNA directing protein as described herein) catalyzes the transposition of the modified transposable element containing the nucleic acid sequence of interest into the genomic DNA of the plant. The present invention therefore increases the efficiency of integration.

For example, for grasses such as maize, the elements of the transposon-based method can be introduced into a cell using, for example, microprojectile bombardment which is incorporated herein by reference in its entirety (see, e.g., Sanford, J. C., et al., U.S. Pat. No. 5,100,792 (1992). In this approach, the elements of the transposon-based compositions are coated onto small particles which are then introduced into the targeted tissue (cells) via high velocity ballistic penetration. The transformed cells are then cultivated under conditions appropriate for the regeneration of plants, resulting in production of transgenic plants. Transgenic plants carrying a nucleic acid sequence of interest are examined for the desired phenotype using a variety of methods including, but not limited to, an appropriate phenotypic marker, such as antibiotic resistance or herbicide resistance, or visual observation of the time of floral induction compared to naturally-occurring plants.

Further, the gene transfer system of this invention can be used as part of a process for working with or for screening a library of recombinant sequences, for example, to assess the function of the sequences or to screen for protein expression, or to assess the effect of a particular protein or a particular expression control region on a particular cell type. In this example, a library of recombinant sequences, such as the product of a combinatorial library or the product of gene shuffling, both techniques now known in the art, can be incorporated into the nucleic acid fragment of this invention to produce a library of nucleic acid fragments with varying nucleic acid sequences positioned between constant inverted repeat sequences.

An advantage of this system is that it is not limited to a significant extent by the size of the intervening nucleic acid sequence positioned between the inverted repeats. For example, the SB protein has been used to incorporate transposons ranging from 1.3 kilobases (kb) to about 5.0 kb and the mariner transposase has mobilized transposons up to about 13 kb. There is no known limit on the size of the nucleic acid sequence that can be incorporated into DNA of a cell using the piggyBac protein.

The transposon-based vectors approach has several advantages over the recombination techniques currently in use such as the Cre/LoxP system. For example, the introduction of nucleic acids sequences of interest is performed directly by the Minos transposon. No additional components, such as target sites, are required. In addition, using the present method, a single copy of a nucleic acid sequence of interest can be integrated and precisely excised from the genetic material of a cell in each integration step.

This invention has significant advantages over current transposon-based vectors vectors for targeted integration (see for example, U.S. Pat. No. 5,958,775 Inventor: E. Wickstrom and Stephen Cleaver; Wickstrom E, et al. Gene (2000) 254:37-44), which describes the uses and limitations of the attTn7 site or of similar sequence which may or may not be similar enough in certain species. The present compositions and methods increase the efficiency of site-selective integration by inserting host-like sequences as described herein. Furthermore, this invention could be used to bypass Tn7 transposase's normal target site(s) by substituting its host DNA directing factor with another. Also, this invention allows for the utilization of the targeting protein of Tn7 (i.e., TnsD) in a simpler and more efficient system, e.g. making a chimeric Tn5-TnsD transposase by recombinant methods described herein.

What has also been limiting the use of transposon-based therapies is the method by which the gene transfer system of this invention is introduced into cells. Viral-mediated strategies have limited the length of the nucleic acid sequence positioned between the inverted repeats, according to this invention. In contrast, for the present non-viral transposon based method microinjection is used and there is very little restraint on the size of the intervening sequence of the nucleic acid fragment of this invention. Similarly, the lipid-mediated strategies described herein for delivering the present nucleic acids do not have substantial size limitations.

There are several combinations of delivery mechanisms for the transposon portion containing the transgene of interest flanked by the inverted terminal repeats (IRs) and the gene encoding the transposase. For example, both the transposon and the chimeric transposase gene can be contained together on the same vector (recombinant viral genome or plasmid); a single infection delivers both parts of the present transposon system such that expression of the transposase then directs cleavage of the transposon from the recombinant viral genome for subsequent integration into a cellular chromosome. In another example, the transposase and the transposon can be delivered separately by a combination of vectors (viruses and/or non-viral systems such as lipid-containing reagents). In these cases either the transposon and/or the transposase gene can be delivered by a recombinant virus. In every case, the expressed transposase gene directs liberation of the transposon from its carrier DNA (viral genome) for site-specific integration into chromosomal DNA.

This invention also relates to compositions for use in the gene transfer system of this invention. Thus, the invention relates to the introduction of a nucleic acid fragment comprising a nucleic acid sequence positioned between at least two inverted repeats into a cell. In a preferred embodiment, efficient incorporation of the nucleic acid fragment into the DNA of into the DNA of a cell occurs when the cell also contains a chimeric transposase as described herein. As discussed above, the chimeric transposase can be provided to the cell as a chimeric transposase or as nucleic acid encoding the chimeric transposase. Nucleic acid encoding the chimeric transposase can take the form of RNA or DNA. The protein can be introduced into the cell alone or in a vector, such as a plasmid or a viral vector. Further, the nucleic acid encoding the chimeric transposase protein can be stably or transiently incorporated into the genome of the cell to facilitate temporary or prolonged expression of the chimeric transposase in the cell. Further, promoters or other expression control regions can be operably linked with the nucleic acid encoding the chimeric transposase to regulate expression of the protein in a quantitative or in a tissue-specific manner. Many transposases have a nuclear localizing signal (NLS). The NLS is required for transport into the nucleus after translation in the cytosol in those cells that are non-dividing. For example, the SB protein contains a DNA-binding domain, a catalytic domain (having transposase activity) and an NLS signal.

The nucleic acid of this invention is introduced into one or more cells using any of a variety of techniques known in the art such as, but not limited to, microinjection, combining the nucleic acid fragment with lipid vesicles, such as cationic lipid vesicles, particle bombardment, electroporation, DNA condensing reagents (e.g., calcium phosphate, polylysine or polyethyleneimine) or incorporating the nucleic acid fragment into a viral vector and contacting the viral vector with the cell. Where a viral vector is used, the viral vector can include any of a variety of viral vectors known in the art including viral vectors selected from the group consisting of a retroviral vector, an adenovirus vector or an adeno-associated viral vector.

P element derived vectors that include at least the P element transposase recognized insertion sequences of the Drosophila P element are provided. As such, this invention includes a pair of the 31 base pair inverted repeat domain of the P element, or the functional equivalent thereof, i.e. a domain recognized by the P element encoded chimeric transposase. The 31 base pair inverted repeat is disclosed in Beall et al., “Drosophila P-element transposase is a novel site-specific endonuclease,” Genes Dev (Aug. 15, 1997) 11(16):2137-51 and incorporated herein by reference. Also incorporated by reference is the amino acid sequence of the P element transposase is disclosed in Rio et al., Cell (Jan. 17, 1986) 44: 21-32).

Non-viral packaging systems (e.g., lipid based, polymer based, lipid-polymer-polymer-based, and polylysine, among others) are well known to those in the field of non-viral transgenic delivery. Further techniques, to augment the delivery into the nucleus are well known and have been employed in non-viral vectors. Methods of assembling in vitro a transposon-transposase complex have been described in the literature and are herein incorporated by reference in their entireity for their teachings on methods of assembling transposon-transposase complexes (Lamberg, A, et al. (2002) Efficient insertion mutagenesis strategy for bacterial genomes involving electroporation of in vitro-assembeled DNA transposition complexes of bacteriophage Mu. Applied and Environmental Microbiology).

Examples of specific ligands for cellular targeting in the packaging system are well known in the art. The following references are incorporated in their entirety for their teachings on specific ligands: (1) Lestina, B. J., Sagnella, S. M., Xu, Z., Shive, M. S., Richter, N. J., Jayaseharan, J., Case, A. J., Kottke-Marchant, K., Anderson, J. M., and Marchant, R. E. (2002) Surface modification of liposomes for selective cell targeting in cardiovascular drug delivery. J. Control Release 78:235-247. (2) Moreira, J. N., Gaspar, R., and Allen, T. M. (2001) Targeting stealth liposomes in a murine model of human small cell lung cancer. Biochim. Biophys. Acta. 1515:167-176; (3) Xu, L., Tang, W. H., Huang, C. C., Alexander, W., Xiang, L. M., Pirollo, K. F., Rait, A., and Chang, E. H. (2001) Systemic p53 gene therapy of cancer with immunolipoplexes targeted by anti-transferrin receptor scFv. Mol. Med. 7:723-734; (4) Sudhan Shaik, M., Kanikkannan, N., and Singh, M. (2001) Conjugation of anti-My9 antibody to stealth monensin liposomes and the effect of conjugated liposomes on the cytotoxicity of immunotoxin. J. Control Realease 76:285-295; (5) Li, X., Stuckert, P., Bosch, I., Marks, J. D., and Marasco, W. A. (2001) Single-chain antibody-mediated gene delivery into ErbB2-positive human breast cancer cells. Cancer Gene Ther. 8:555-565; (6) Park, J. W., Kirpotin, D. B., Hong, K., Shalaby, R., Shao, Y., Nielsen, U. B., Marks, J. D., Papahadjopoules, D., and Benz, C. C. (2001) Tumor targeting using anti-her2 immunoliposomes. J. Control Release 74:95-113.

Examples of endosomal disruption factors that are used in the present vector packaging are well known in the art. The following references are incorporated in their entirety for their teachings on endosomal disruption factors: (1) Farhood, H., Gao, X., Son, K., Yang, Y. Y., Lazo, J. S., Huang, L., Barsoum, J., Bottega, R., and Epand, R. M. (1994) Cationic liposomes for direct gene transfer in therapy of cancer and other diseases. Ann. NY Acad. Sci. 716:23-35; (2) Tachibana R, Harashima H, Shono M, Azumano M, Niwa M, Futaki S, and Kiwada H. (1998) Intracellular regulation of macromolecules using pH-sensitive liposomes and sensitive liposomes and nuclear localization signal: qualitative and quantitative evaluation of intracellular trafficking. Biochem. Biophys. Res. Commun. 251:538-544; (3) El Ouahabi A, Thiry M, Pector V, Fuks R, Ruysschaert J M, and Vandenbranden M. (1997) The role of endosome destabilization activity in the gene transfer process mediated by cationic lipids. FEBS Lett 414:187-192.

Nuclear localization factors for use in delivering the present vectors are well known in the art. The following references are incorporated in their entirety for their teachings on nuclear localization factors: (1) Subramanian A, Ranganathan P, and Diamond SL. (1999) Nuclear targeting peptide scaffolds for lipofection of nondividing mammalian cells. Nat Biotechnol 17:873-877; (2) Tachibana R, Harashima H, Shono M, Azumano M, Niwa M, Futaki S, and Kiwada H. (1998) Intracellular regulation of macromolecules using pH-sensitive liposomes and nuclear localization signal: qualitative and quantitative evaluation of intracellular trafficking. Biochem. Biophys. Res. Commun. 251:538-544. (3) Aronsohn A I and Hughes J A. (1998) Nuclear localization signal peptides enhance cationic liposome-mediated gene transfer. J Drug Target 5:163-169; (4) Boehm U, Heinlein M, Behrens U, and Kunze R. (1995) One of three nuclear localization signals of maize Activator (Ac) transposase overlaps the DNA-binding domain. Plant J 7:441-451.

Also disclosed are compositions of the invention, wherein the integrating enzyme is located outside the terminal repeats.

Also disclosed are compositions of the invention, wherein the transgene and the integrating enzyme are encoded on the same nucleic acid.

Also disclosed are compositions of the invention, wherein the transgene and the integrating enzyme are encoded on a separate nucleic acids.

Also disclosed are compositions of the invention, further comprising a homologous sequence that is homologous to the host DNA.

Also disclosed are compositions of the invention, wherein the homologous sequence is located outside the terminal repeats.

Also disclosed are compositions of the invention, further comprising a protein binding sequence and a separate nucleic acid encoding two DNA binding domains.

Also disclosed are compositions of the invention, further comprising a protein binding sequence and a separate nucleic acid encoding a DNA binding domain and a protein-binding domain.

Also disclosed are compositions of the invention, wherein the nucleic acid present in the non-viral vector is at least one functional protein.

Also disclosed are compositions of the invention, wherein the transgene encodes a biologically active molecule. The transgene can encode multiple and different biologically active molecules. The biologically active molecules can be therapeutic. The transgene can be selected at least from the group consisting of reporter genes (e.g., luciferase, chloramphenicol-acetyl transferase, GFP), oncogenes (e.g., ras and c-myc), and antioncogenes (e.g. p53 and retinoblastoma). A variety of other genes are being tested for gene therapy including CFTR for cystic fibrosis, adenosine deaminase (ADA) for immune disorders, factor IX, factor VIII and interleukin-2 (IL-2) for blood cell diseases, alpha-1-antitrypsin for lung disease, and tumor necrosis factor, endostatin, sodium/iodide symporter, angiostatin, and multiple drug resistance (MDR) for cancer therapies. Other examples of genes include, e.g., bax, bak, E2F-1, BRCA-1, BRCA-2, bak, ras, p21, CDKN2A, pHyde, FAS-ligand, TNF-related apoptosis inducing ligand, DOC-2, E-cadherin, caspases, clusterin, ATM, granulocyte macrophage colony stimulating factor, B7, tumor necrosis factor-alpha, interleuken 12, interleuken 15, interferon-gamma, interferon-beta, MUC-1, PSA, WT1, WT2, myc, MDM2, DCC, VEGFB, VEGFC, VWF, NEFL, NEF3, TUBB, MAPT, SGNE1, RTN1, GAD1, PYGM, AMPD1, TNNT3, TNNT2, ACTC, MYH7, SFTPB, TPO, NGF, connexin 43.

Compounds disclosed herein can also be used for the treatment of precancer conditions such as cervical and anal dysplasias, other dysplasias, severe dysplasias, hyperplasias, atypical hyperplasias, and neoplasias.

Also disclosed are vectors of the invention, wherein the transgene is an antigen from a virus. The viral antigen can be selected from the group consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

Also disclosed are vectors of the invention, wherein the transgene is an antigen from a bacterium. The bacterial antigen can be selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Also disclosed are vectors of the invention, wherein the transgene is antigen from a parasite. The parasitic antigen can be selected from the group consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

Also disclosed are vectors of the invention, wherein the transgene is a tumor antigen. The tumor antigen can be selected from the list consisting of human epithelial cell mucin (Muc-1; a 20 amino acid core repeat for Muc-1 glycoprotein, present on breast cancer cells and pancreatic cancer cells), the Ha-ras oncogene product, p53, carcino-embryonic antigen (CEA), the raf oncogene product, gp100/pmel17, GD2, GD3, GM2, TF, sTn, MAGE-1, MAGE-3, BAGE, GAGE, tyrosinase, gp75, Melan-A/Mart-1, gp100, HER2/neu, EBV-LMP 1 & 2, HPV-F4, 6, 7, prostate-specific antigen (PSA), HPV-16, MUM, alpha-fetoprotein (AFP), CO17-1A, GA733, gp72, p53, the ras oncogene product, HPV E7, Wilm's tumor antigen-1, telomerase, tumor antigen-1, telomerase, and melanoma gangliosides.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular chimeric transposase is disclosed and discussed and a number of modifications that can be made to a number of molecules including the chimeric transposase are discussed, specifically contemplated is each and every combination and permutation of chimeric transposase and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Also disclosed are methods of docking the transposon-based vector adjacent to the host DNA, utilizing repetitive sequences for homologous recombination to promote efficient site-selective integration, as well as other site-selective non-viral approaches.

Also disclosed are methods that employ recognition site(s) on the plasmid that can recognize an endogenous protein (or a newly introduced protein, e.g. produced from a gene located on the plasmid) that can then direct the complex into the vicinity of the host-DNA for site-selective integration.

Also disclosed are methods of incorporating repetitive elements (e.g., Alu-like sequences) in the transposon-based plasmid. It is understood that such methods can enhance docking and at the same time allow for either homologous recombination (66-67) or integration of the transgene into the host genome.

Incorporating repetitive elements (e.g., Alu-like sequences) in the transposon-based plasmid can enhance docking and at the same time allow for either homologous recombination or integration of the transgene into the host genome.

Also disclosed are methods that employ recognition sites on the plasmid that can recognize an endogenous protein (or a newly introduced protein) that can then direct the complex to the vicinity of the host-DNA.

1. Delivery of the Vector Compositions to Cells

There are a number of compositions and methods which can be used to deliver nucleic acids to cells, either in vitro or in vivo. For example, the nucleic acids can be delivered through a number of direct delivery systems such as, electroporation, lipofection, calcium phosphate precipitation, plasmids, cosmids, or via transfer of genetic material in cells or carriers such as cationic liposomes. Appropriate means for transfection, including chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed non-viral vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of ultrasound mediated delivery, the technology for which is available from multiple vendors including but not limited to the SONOPORATION machine, which is available from ImaRx Pharmaceutical Corp. (Tucson, Ariz.).

The materials may be in solution, suspension (for example, incorporated into microparticles, liposomes, or cells). These can be targeted to a particular cell type via antibodies, receptors, or receptor ligands. The following references are examples of the use of this technology to target specific proteins to tumor tissue and are incorporated by reference herein (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue and are incorporated by reference herein (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

The 3 requirements for efficient cell-selective delivery of a vector into the nucleus of a cell are a ligand (or receptor) for selective cell targeting, an endosomal disruption factor if the vector is taken up via receptor mediated endocytosis, and a nuclear localizing signal. These have been employed in gene therapy and the methods of construction and implementation are well known in the literature.

Surface modifications to liposomes for selective cell targeting have been described in detail and employed with success and are incorporated by reference herein (Lestini, B. J., et al (2002) Surface modification of liposomes for selective cell targeting in cardiovascular drug delivery. J. Control Release 78:235-247; Moreira, J. N., et al. (2001) Targeting stealth liposomes in a murine model of human small cell lung cancer. Biochim. Biophys. Acta. 1515:167-176.; Xu, L., et al. (2001) Systemic p53 gene therapy of cancer with immunolipoplexes targeted by anti-transferrin receptor scFv. Mol. Med. 7:723-734. Sudhan Sudhan Shaik, M., et al. (2001) Conjugation of anti-My9 antibody to stealth monensin liposomes and the effect of conjugated liposomes on the cytotoxicity of immunotoxin. J. Control Realease 76:285-295.; Li, X., et al. (2001) Single-chain antibody-mediated gene delivery into ErbB2-positive human breast cancer cells. Cancer Gene Ther. 8:555-565.; Park, J. W., et al. (2001) Tumor targeting using anti-her2 immunoliposomes. J. Control Release 74:95-113). For example, a cationic immunolipolex incorporating a biosynthetically lipid-tagged, anti-transferrrin receptor could be utilized as described by Xu and colleagues.

Endosomal disruption factors have been employed in cationic lipids and are well known to those who are skilled in the art (Tachibana R, et al. (1998) Intracellular regulation of macromolecules using pH-sensitive liposomes and nuclear localization signal: qualitative and quantitative evaluation of intracellular trafficking. Biochem. Biophys. Res. Commun. 251:538-544; El Ouahabi A, et al. (1997) The role of endosome destabilization activity in the gene transfer process mediated by cationic lipids. FEBS Lett 414:187-192). For example, Tachibana and colleagues utilized pH-sensitive liposomes in order to achieve endosomal disruption and subsequent release into the cytosol.

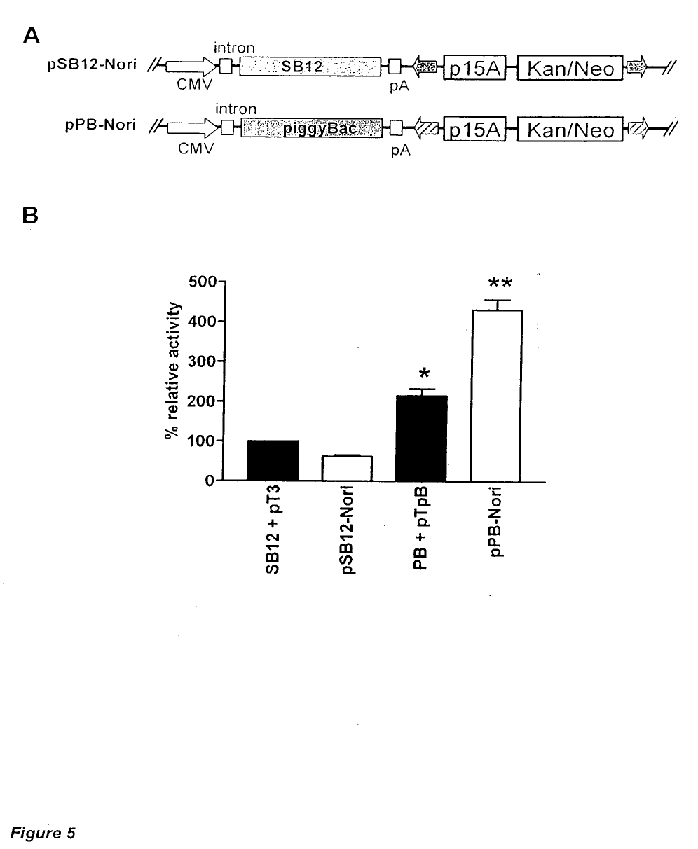

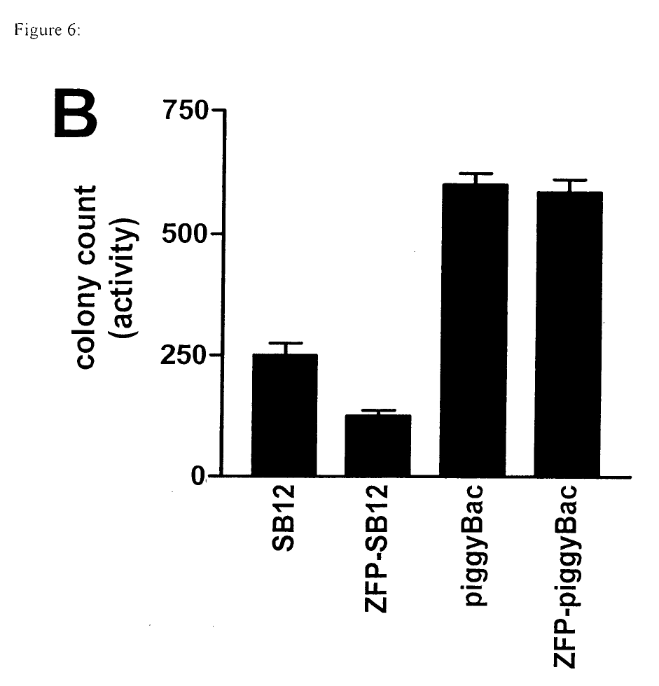

Nuclear localization factors can also be incorporated as diagrammed in the schematic (FIGS. 5 and 6) (Subramanian A, et al. (1999) Nuclear targeting peptide scaffolds for lipofection of nondividing mammalian cells. Nat Biotechnol 17:873-877.; Aronsohn A I, et al. (1998) Nuclear localization signal peptides enhance cationic liposome-mediated gene transfer. J Drug Target 5:163-169.; Boehm U, et al. (1995) One of three nuclear localization signals of maize Activator (Ac) transposase overlaps the DNA-binding domain. Plant J 7:441-451.) For example, Aronsohn and colleagues constructed a non-viral delivery vehicle consisting of a conglomerate of a synthetic nuclear localizing peptide derived from the SV40 virus, a luciferase encoding PGL3 plasmid, and a cationic lipid DOTAP:DOPE liposome.

2. Expression Systems

The nucleic acids that are delivered to cells typically contain expression controlling systems. For example, the inserted genes in non-viral and viral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Promoters and Enhancers

Preferred promoters controlling transcription from vectors in mammalian host cells can be obtained from various sources, for example, the genomes of viruses such as: cytomegalovirus, polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

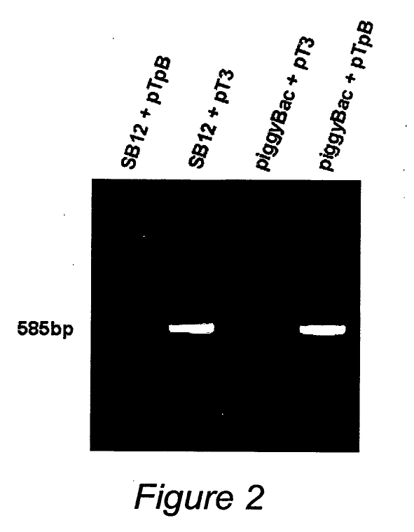

Inducible promoters can be “turned on” in response to an exogenously supplied agent or stimulus, which is generally not an endogenous metabolite or cytokine. Inducible promoters include those using the lac repressor from E. coli as a transcription modulator to modulator to regulate transcription from lac operator-bearing mammalian cell promoters [Brown, M. et al., Cell, 49:603-612 (1987)], those using an antibiotic-inducible promoter, such as the tetracycline repressor (tetR) [Gossen, M., and Bujard, H., Proc. Natl. Acad. Sci USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. See Miller, N. and Whelan, J., Human Gene Therapy, 8:803-815 (1997). Other systems include FK506 dimer, VP16 or p65 using astradiol, RU486, diphenol murislerone or rapamycin [see Miller and Whelan, supra at FIG. 2]. Inducible systems are available from Invitrogen, Clontech and Ariad. Systems using a repessor with the operon are preferred. Regulation of transgene expression in target cells represents a critical aspect of gene therapy. For example, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetr) with the transcription activator (VP16) to create a tetR-mammalian cell transcription activator fusion protein, tTa (tetR-VP 16), with the teto-bearing minimal promoter derived from the human cytomegalovirus (hCMV) major immediate-early promoter to create a tetR-tet operator system to control gene expression in mammalian cells. Recently Yao and colleagues [F. Yao et al., Human Gene Therapy, supra] demonstrated that the tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter. One particular advantage of this tetracycline inducible switch is that it does not require the use of a tetracycline repressor-mammalian cells transactivator or repressor fusion protein, which in some instances can be toxic to cells [M. Gossen et al., Natl. Acad. Sci. USA, 89:5547-5551 (1992); P. Shockett et al., Proc. Natl. Acad. Sci. USA, 92:6522-6526 (1995)], to achieve its regulatable effects. The repressor can be linked to the target molecule by an IRES sequence. The inducible system can be a tetR system. If so, the system can have the tetracycline operation downstream of a promoter's TATA element such as with the CMVIE promoter.

Further examples of inducible promoters or other gene regulatory elements include a heat-inducible promoter, a light-inducible promoter, or a laser inducible promoter (e.g., Halloran et al. (2000) Development 127: 1953-1960; Gemer et al. (2000) Int. J. Hyperthermia Hyperthermia 16: 171-81; Rang and Will, 2000, Nucleic Acids Res. 28: 1120-5; Hagihara et al. (1999) Cell Transplant 8: 4314; Huang et al. (1999) Mol. Med. 5: 129-37; Forster et al. (1999) Nucleic Acids Res. 27: 708-10; Liu et al. (1998) Biotechniques 24: 624-8, 630-2; the contents of which have been incorporated herein by reference in their entireties); metallothionein promoter, ecdysone, and other steroid-responsive promoters, rapamycin responsive promoters, and the like (No et al., Proc. Natl. Acad. Sci. USA, 93:3346-51 (1996); Furth et al., Proc. Natl. Acad. Sci. USA, 91:9302-6 (1994), incorporated herein by reference for their teaching of inducible promoters and their uses). Additional control elements that can be used include promoters requiring specific transcription factors such as viral promoters. The present piggyBac vectors can be used to investigate the use of other drug-mediated-dimerization responsive promoters and other means of achieving titratable gene expression in vivo.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Suitable promoters for use in plants are also well known in the art. For example, constitutive promoters for plant gene expression include the octopine synthase, nopaline synthase, or mannopine synthase promoters from Agrobacterium, the cauliflower mosaic virus (35S) promoter, the figwort mosaic virus (FMV) promoter, and the tobacco mosaic virus (TMV) promoter. Specific examples of regulated promoters in plants are incorporated herein by reference include the low temperature Kin1 and cor6.6 promoters (Wang, et al., Plant Mol. Biol. 28:605 (1995); Wang, et al., Plant Mol. Biol. 28:619-634 (1995)), the ABA inducible promoter (Marcotte et al., Plant Cell 1:969-976 (1989)), heat shock promoters, and the cold inducible promoter from B. napus (White et al., Plant Physiol. 106:917 (1994)).

Expression vectors used in eukaryotic host cells (yeast, fungi, insect, plant, animal, human or nucleated cells) can also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The vector can include nucleic acid sequence encoding a marker product. The term “marker gene”, as used herein, refers to a nucleic acid sequence whose product can be easily assayed, for example, colorimetrically as an enzymatic reaction product, such as the lacZ gene which encodes for .beta.-galactosidase. The marker gene can be operably linked to a suitable promoter which is optionally linked to a nucleic acid sequence of interest so that expression of the marker gene can be used to assay integration of the transposon into the genome of a cell and thereby integration of the nucleic acid sequence of interest into the genome of the cell. Examples of widely-used marker molecules include enzymes such as beta-galactosidase, beta-glucoronidase, beta-glucosidase; luminescent molecules such as green flourescent protein and firefly luciferase; and auxotrophic markers such as His3p and Ura3p. (See, e.g., Chapter 9 in Ausubel, F. M., et al. Current Protocols in Molecular Biology, John Wiley & Sons, Inc., (1998)).

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### C. METHODS OF USING THE COMPOSITIONS

The transposon system of this invention has applications to many areas of biotechnology. Development of transposable elements for vectors in animals permits the following: 1) efficient insertion of genetic material into animal chromosomes using the methods given in this application; 2) identification, isolation, and characterization of genes involved with growth and development through the use of transposons as insertional mutagens (e.g., see Kaiser et al., 1995, “Eukaryotic transposable elements as tools to study gene structure and function.” In Mobile Genetic Elements, IRL Press, pp. 69-100); 3) identification, isolation and characterization of transcriptional regulatory sequences controlling growth and development; 4) use of marker constructs for quantitative trait loci (QTL) analysis; and 5) identification of genetic loci of economically important traits, besides those for growth and development, i.e., disease resistance (e.g., Anderson et al., 1996, Mol. Mar. Biol. Biotech., 5, 105-113).

1. Methods of Gene Modification and Gene Disruption

Due to their inherent ability to move from one chromosomal location to another another within and between genomes, transposable elements have been exploited as genetic vectors for genetic manipulations in several organisms. Transposon tagging is a technique in which transposons are mobilized to “hop” into genes, thereby inactivating them by insertional mutagenesis. These methods are discussed by Evans et al., TIG 1997 13, 370-374. In the process, the inactivated genes are “tagged” by the transposable element which then can be used to recover the mutated allele. The ability of the human and other genome projects to acquire gene sequence data has outpaced the ability of scientists to ascribe biological function to the new genes. Therefore, the present invention provides an efficient method for introducing a tag into the genome of a cell. Where the tag is inserted into a location in the cell that disrupts expression of a protein that is associated with a particular phenotype, expression of an altered phenotype in a cell containing the nucleic acid of this invention permits the association of a particular phenotype with a particular gene that has been disrupted by the nucleic acid fragment of this invention. Here the nucleic acid fragment functions as a tag. Primers designed to sequence the genomic DNA flanking the nucleic acid fragment of this invention can be used to obtain sequence information about the disrupted gene.

The nucleic acid fragment can also be used for gene discovery. In one example, the nucleic acid fragment in combination with the chimeric transposase or nucleic acid encoding the chimeric transposase is introduced into a cell. The nucleic acid fragment preferably comprises a nucleic acid sequence positioned between at least two inverted repeats, wherein the inverted repeats bind to the chimeric transposase protein and wherein the nucleic acid fragment integrates into the DNA of the cell in the presence of the chimeric transposase protein. In a preferred embodiment, the nucleic acid sequence includes a marker protein, such as GFP and a restriction endonuclease recognition site, preferably a 6-base recognition sequence. Following integration, the cell DNA is isolated and digested with the restriction endonculease. Where a restriction endonuclease is used that employs a 6-base recognition sequence, the cell DNA is cut into about 4000-bp fragments on average. These fragments can be either cloned or linkers can be added to the ends of the digested fragments to provide complementary sequence for PCR primers. Where linkers are added, PCR reactions are used to amplify fragments using primers from the linkers and primers binding to the direct repeats of the inverted repeats in the nucleic acid fragment. The amplified fragments are then sequenced and the DNA flanking the direct repeats is used to search computer databases such as GenBank.

In another application of this invention, the invention provides a method for delivering a transgene to a cell by transfecting or transforming the cell with a vector that expresses a transposase, and a vector that contains a natural or synthetic transposable element.

Provided is a method for the delivery of a gene regulatory sequence that drives expression of a marker gene or in order to evaluate the properties of that gene regulatory sequence. The method generally comprises delivering the gene regulatory sequence (e.g., unknown/uncharacterized promoter) functionally linked to the marker gene in a transcriptional unit, to a cellular or animal system in which expression of the marker gene can be detected. The method can be used to assess regulatory sequence function, including but not limited to which sequences confer tissue specificity or which cell types express a given amount of a specific DNA binding protein. In another application, the compositions and methods provide a method for integrating at multiple copies of the same transgene in a cell. For example, in some embodiments the number of copies of transgenes in a cell can be from at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35, 36, 37, 38, 39, 40, 41, 42, 43, 44, 45, 46, 47, 48, 49 or 50. Thus, not only does the method encompass integration at each specified level, but at any point within the recited range of 3-50 copies.

In another application of this invention, the invention provides a method for delivering a transgene to a cell by transfecting or transforming the cell with a vector that expresses a transposase, and a vector that contains a natural or synthetic transposable element that expresses multiple genes via a bicistronic mRNA.

In another application of this invention, the invention provides a method for integrating inducible promoters that are multi-component systems, including but not limited to the tet on and tet off systems, and ecdysone system as further described herein. The present mult-gene vectors are particularly well-suited to these inducible systems, because they permit concurrent expression of the multiple components required by these systems.

In another application of this invention, the invention provides a method for integrating two or more different transgenes per cell.

In another application of this invention, the invention provides a method for delivering a transgene to a cell comprising transfecting or transforming cell with a vector that both expresses a transposase and encodes a natural or synthetic transposon.

In another application of this invention, the invention provides a method for overcoming overproduction inhibition by delivering a transgene that expresses the piggyBac transposase.

In another application of this invention, the invention provides a method for maintaining piggyBac activity in a cell despite the covalent addition of a zinc finger DNA binding domain to the transposase by transfecting or transforming a cell with a vector that expresses the chimeric protein.

Also provided is a method for delivering multiple genes to a cell, comprising delivering to the cell a nucleic acid comprising a first minimal piggyBac inverted repeat element, a promoter (constitutive or inducible), a gene coding sequence (transcriptional unit) or region to receive a gene coding sequence (transcriptional unit), a IRES element, a second gene coding sequence or region to receive a gene coding sequence, and a second minimal piggyBac inverted repeat element. The method can be used for a number of purposes including but not limited to multi-gene transfer in mammals and other species, reconstitution of signaling pathways, multiple different marker genes, creation of multi-gene transgenic animals, gene therapy for single and multi-gene disorders, evaluation of multiple different promoters simultaneously, simultaneous multi-gene knockout/mutagenesis, the formation of stable packaging cells for viruses, and drug discovery applications.

Internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988, incorporated herein by reference for the teaching of IRES sequences and their positioning). IRES elements from two members of the picornavirus family (polio and encephalomyocarditis) have been described (Pelletier and Sonenberg, 1988), as well an IRES from a mammalian message (Macejak and Sarnow, 1991, incorporated herein by reference for the teaching of IRES sequences and their positioning). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (U.S. Pat. Nos. 5,925,565 and 5,935,819; PCT/US99/05781, incorporated herein by reference for their teaching of IRES sequences and their positioning). IRES sequences are known in the art and include those from encephalomycarditis virus (EMCV) [Ghattas, I. R. et al., Mol. Cell. Biol., al., Mol. Cell. Biol., 11:5848-5849 (1991), incorporated herein by reference for the teaching of IRES sequences and their positioning; BiP protein [Macejak and Sarnow, Nature, 353:91 (1991), incorporated herein by reference for the teaching of IRES sequences and their positioning]; the Antennapedia gene of drosophilia (exons d and e) [Oh et al., Genes & Development, 6:1643-1653 (1992), incorporated herein by reference for the teaching of IRES sequences and their positioning]; those in polio virus [Pelletier and Sonenberg, Nature, 334:320325 (1988); see also Mountford and Smith, TIG, 11: 179-184 (1985), incorporated herein by reference for the teaching of IRES sequences and their positioning].

The present piggyBac multi-gene compositions and methods can be used build cell lines for use in drug discover. For example provided are methods and compositions to simultaneously deliver mGluR3 with a glutamate transporter (GLAST, which is necessary to remove glutamate from outside of the cell for the receptor to be activated), a promiscuous G-protein, and a G-protein activated potassium channel (GirK2). Currently, there is not a suitable cell line for drug discovery for mGluR3 due to the lack of cellular components necessary to evaluate drugs directed at this receptor. Successful reconstitution of this signaling pathway in cells (4 genes total) is easily detectable measuring Ca⁺⁺ mobilization with the use of Ca⁺⁺ sensitive fluorescent dyes. Selection followed by cell sorting into 96 well plates will permit isolation of cell clones. These clones are then be tested with the use of a glutamate receptor agonist to check for mGluR3 dependent Ca⁺⁺ mobilization. Clonal cells will only fluoresce if they have taken up all the necessary components permitting easy selection of cells containing all of the necessary signaling molecules. These cell lines can then be expanded and used in high throughput screening (HTS) assays for development of receptor specific antagonists (blockers) or agonists (activators). This one example shows the power of piggyBac in genetically engineering cells for a specific phenotype, in this case reconstitution of a specific signaling pathway for drug discovery applications. The methods and compositions can be used to genetically engineer T-cells for immunotherapy and stem cells for therapeutic applications using piggyBac.

The disclosed methods and compositions can be used for site-directed tagging. For example, by incorporating a similiar host gene sequence (but non-functional) in a transposon based plasmid allows for tagging of that gene as described above. One application of the invention is to determine the function of a specific protein. For example, cDNA (reverse transcribed mRNA), genomic DNA, or RNA/DNA hybrids (chimeraplast) can be inserted in a transposon-based palsmid after site-directed mutagenesis so that the coding region can be coding region can be inactivated. This altered cDNA or genomic DNA can be inserted into a tranposon-based plasmid as described herein. The transposon-based vector containing host-like sequence docks to the host DNA through hybridization. Expression of the transposase and subsequent integration occurs at the desired target. Another embodiment of the invention is making a chimeric transposase without site-selectivity for the purposes described above. For example, if a given transposase in a certain cell does not have the DNA directing factor for that cell then the efficiency of integration is markedly reduced. By providing the transposase with a required DNA directing factor then the integration is significantly enhanced which results in an obvious improvement over the “conventional” transposase.

In another application of this invention, the invention provides a method for mobilizing a nucleic acid sequence in a cell. In this method the nucleic acid fragment of this invention is incorporated into DNA in a cell, as provided in the discussion above. Additional chimeric transposase or nucleic acid encoding the chimeric transposase is introduced into the cell and the protein is able to mobilize (i.e. move) the nucleic acid fragment from a first position within the DNA of the cell to a second position within the DNA of the cell. The DNA of the cell can be genomic DNA or extrachromosomal DNA. The method permits the movement of the nucleic acid fragment from one location in the genome to another location in the genome, or for example, from a plasmid in a cell to the genome of that cell.

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination events occur at a low frequency.

Once the cell is produced through the methods described herein, an animal can be produced from this cell through either stem cell technology or cloning technology. For example, if the cell into which the nucleic acid was transfected was a stem-cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

To modify a gene of interest nucleic acids can be cloned into a vector designed for example, for homologous recombination. This gene could be, for example, a heterologous or synthetic regulatory sequence of an antioncogene (e.g. p53 and retinoblastoma). A variety of other genes are being tested for gene therapy including CFTR for cystic fibrosis, adenosine deaminase (ADA) for immune disorders, factor IX, factor VIII and interleukin-2 (IL-2) for blood cell diseases, alpha-1-antitrypsin for lung disease, and tumor necrosis factor, endostatin, sodium/iodide symporter, angiostatin, and multiple drug resistance (MDR) for cancer therapies. Other examples gene include e.g., bax, bak, E2F-1, BRCA-1, BRCA-2, bak, ras, p21, CDKN2A, pHyde, FAS-ligand, TNF-related apoptosis inducing ligand, DOC-2, E-cadherin, caspases, clusterin, ATM, granulocyte macrophage colony stimulating factor, B7, tumor necrosis factor-alpha, interleuken 12, interleuken 15, interferon-gamma, interferon-beta, MUC-1, PSA, WT1, WT2, myc, MDM2, DCC, VEGFB, VEGFC, VWF, NEFL, NEF3, TUBB, MAPT, SGNE1, RTN1, GAD1, PYGM, AMPD1, TNNT3, TNNT2, ACTC, MYH7, SFTPB, TPO, NGF, connexin 43.

2. Methods of Performing Gene Delivery

Gene delivery is performed in vitro (e.g., electroporation or other techniques well known in the art) or in vivo. In vivo techniques include intravenous administration, direct injection into the desired site, or by inhalation.

3. Methods of Treating Disease

Disclosed are methods of treating a subject with a condition comprising administering to the subject a vector of the invention.

The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general. Cancer therapeutic genes that can be delivered via the subject vectors include: genes that enhance the antitumor activity of lymphocytes, genes whose expression product enhances the immunogenicity of tumor cells, tumor suppressor genes, toxin genes, suicide genes, multiple-drug resistance genes, antisense sequences, small interfering RNAs and the like.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Also disclosed are methods of the invention, wherein the transgene is a tumor antigen. The tumor antigen can be selected from the list consisting of human epithelial cell mucin (Muc-1; a 20 amino acid core repeat for Muc-1 glycoprotein, present on breast cancer cells and pancreatic cancer cells), the Ha-ras oncogene product, p53, carcino-embryonic antigen embryonic antigen (CEA), the raf oncogene product, gp100/pmel17, GD2, GD3, GM2, TF, sTn, MAGE-1, MAGE-3, BAGE, GAGE, tyrosinase, gp75, Melan-A/Mart-1, gp100, HER2/neu, EBV-LMP 1 & 2, HPV-F4, 6, 7, prostate-specific antigen (PSA), HPV-16, MUM, alpha-fetoprotein (AFP), CO17-1A, GA733, gp72, p53, the ras oncogene product, HPV E7, Wilm's tumor antigen-1, telomerase, and melanoma gangliosides.

Also disclosed are methods of the invention, wherein the condition is a viral infection. The viral infection can be selected from the list of viruses consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

Also disclosed are methods of the invention, wherein the transgene is an antigen from a virus. The viral antigen can be selected from the group of viruses consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

Also disclosed are methods of the invention, wherein the condition is a bacterial infection. The bacterial infection can be selected from the list of bacterium consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Also disclosed are methods of the invention, wherein the transgene is an antigen from a bacterium. The bacterial antigen can be selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Also disclosed are methods of the invention, wherein the condition is a parasitic parasitic infection. The parasitic infection can be selected from the list of parasites consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

Also disclosed are methods of the invention, wherein the transgene is an antigen from a parasite. The parasitic antigen can be selected from the group consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

Disclosed are methods of treating a condition in a subject comprising administering to the subject the vector of the invention, wherein the condition is due to a mutated, disregulated, disrupted, or deleted gene; autoimmunity; or inflammatory diseases.

Thus, in yet another use of the gene transfer system of this invention, the nucleic acid includes a gene to provide a gene therapy to a cell. The gene is placed under the control of a tissue specific promoter or of a ubiquitous promoter or one or more other expression control regions for the expression of a gene in a cell in need of that gene. Therapeutic nucleic acids of interest include genes that replace defective genes in the target host cell, such as those responsible for genetic defect based diseased conditions, genes which have therapeutic utility in the treatment of cancer, and the like. A variety of genes are being tested for a variety of gene therapies including, but not limited to, the cystic fibrosis transmembrane regulator (CFTR) gene, adenosine deaminase (ADA) for immune system disorders, factor IX and interleukin-2 (IL-2) for blood cell diseases, alpha-1-antitrypsin for lung disease, and tumor necrosis factors (TNFs) and multiple drug resistance (MDR) proteins for cancer therapies. Other specific therapeutic genes for use in the treatment of genetic defect based disease conditions include genes encoding the following products: factor VIII, beta.-globin, low-density protein receptor, purine nucleoside phosphorylase, sphingomyelinase, glucocerebrosidase, cystic fibrosis transmembrane regulator, CD-18, ornithine transcarbamylase, arginosuccinate synthetase, phenylalanine hydroxylase, branched-chain .alpha.-ketoacid dehydrogenase, fumarylacetoacetate hydrolase, glucose 6-phosphatase, .alpha.-L-fucosidase, .beta.-glucuronidase, .alpha.-L-iduronidase, galactose 1-phosphate uridyltransferase, and the like. Because of the length of nucleic acid that can be carried by the subject vectors, the subject vectors can be used to not only introduce a therapeutic gene of interest, but also any expression regulatory elements, such as promoters, and the like, which may be desired so as to obtain the desired temporal and spatial expression of the therapeutic gene. These and a variety of human or animal specific gene sequences including gene sequences to encode marker proteins and a variety of recombinant proteins are available in the known gene databases such as GenBank, and the like.

Disclosed are methods of treating a condition in a subject, wherein the condition can be selected from list consisting of cystic fibrosis, asthma, multiple sclerosis, muscular dystrophy, diabetes, tay-sachs, spinobifida, sickle cell anemia, hereditary hemochromatosis, cerebral palsy, parkinson's disease, lou gehrigg disease, alzheimer's, systemic lupus erythamatosis, hemophelia, Addsion's disease, Huntington's disease, and Cushing's disease.

Disclosed are methods of treating a condition, wherein the transgene is comprises a functioning gene to replace a mutated gene associated with a genetic disorder. Also disclosed are methods of treating a condition, wherein the transgene can be selected from the list of genes consisting of cystic fibrosis transmembrane conductance regulator, HFE, and HBB.

The invention can be particularly useful for vaccine delivery. In this aspect of the invention, the antigen or immunogen can be expressed heterologously (e.g., by recombinant insertion of a nucleic acid sequence which encodes the antigen) or as an immunogen (including antigenic or immunogenic fragments) in a viral vector. Alternatively, the antigen or immunogen can be expressed in a live attenuated, pseudotyped virus vaccine, for example. It is also understood that the non-viral vectors disclosed herein can be used for vaccine delivery. Generally, the methods can be used to generate humoral and cellular immune responses, e.g. via expression of heterologous pathogen-derived proteins or fragments thereof in specific target cells.

4. Pharmaceutical Carriers/Pharmaceutical Delivery

As described above, the compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Delivery for Therapeutic Uses

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The in the art. The dosage can be adjusted by the individual physician in the event of any counterindications. Dosage can vary, and can be administered in one or more dose administrations daily, for one or several days.

Other vectors which do not have a specific pharmacuetical function, but which can be used for tracking changes within cellular chromosomes or for the delivery of diagnostic tools for example can be delivered in ways similar to those described for the pharmaceutical products.

The non-viral vectors of the invention can also be used for example as tools to isolate and test new drug candidates for a variety of diseases. They can also be used for the continued isolation and study, for example, the cell cycle. There use as exogenous DNA delivery devices can be expanded for nearly any reason desired by those of skill in the art.

5. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

6. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA: DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or or method meets any one of these criteria for determining hybridization either collectively or singly it is a composition or method that is disclosed herein.

7. Nucleic Acids

There are a variety of molecules disclosed herein that are nucleic acid based, including for example the nucleic acids that encode, for example a chimeric transposase, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) In Vivo/Ex Vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

8. Peptides

a) Protein Variants

As discussed herein there are numerous variants of the chimeric integrating enzymes and that are known and herein contemplated. In addition, there are derivatives of the chimeric integrating enzymes which also function in the disclosed methods and compositions. Protein variants and derivatives are well understood to those of skill in the art and in can involve amino acid sequence modifications. For example, amino acid sequence modifications typically modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| Alanine | Ala; A |
| allosoleucine | AIle |
| Arginine | Arg; R |
| Asparagine | Asn; N |
| aspartic acid | Asp; D |
| Cysteine | Cys; C |
| glutamic acid | Glu; E |
| Glutamine | Gln; Q |
| Glycine | Gly; G |
| Histidine | His; H |
| Isolelucine | Ile; I |
| Leucine | Leu; L |
| Lysine | Lys; K |
| phenylalanine | Phe; F |
| Proline | Pro; P |
| pyroglutamic acidp | Glu |
| Serine | Ser; S |
| Threonine | Thr; T |
| Tyrosine | Tyr; Y |
| Tryptophan | Trp; W |
| Valine | Val; V |

TABLE 2
Amino Acid Substitutions
Original Residue Exemplary Conservative Substitutions, others are
known in the art.

Ala; Ser
Arg; Lys, Gln
Asn; Gln; His
Asp; Glu
Cys; Ser
Gln; Asn, Lys
Glu; Asp
Gly; Pro
His; Asn; Gln
Ile; Leu; Val
Leu; Ile; Val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode a chimeric transposase obtained from linking a transposase [e.g. Tc1 (Reference No. NM_{—}061407, AI878683, AI878522, AI794017); P-element (Rio et al., Cell (1986) 44:21-32; among others)] to a DNA directing factor [e.g., LexA DBD (Accession No. J01643-V0029-V00300, Hin DNA binding domain (Reference No. J03245), STF-1 DNA binding domain (Reference No. S67435, corresponding to a.a. 140-215 described in Leonard et al. (1993) Mol. Endo. 7:1275-1283), among others]. The sequences can be obtained at Entrez Nucleotide Database, or GenBank or other nucleotide or protein search engines.

9. Kits

Disclosed herein are kits that are drawn to reagents that can be used in practicing the methods disclosed herein. The kits can include any reagent or combination of reagents discussed herein or that would be understood to be required or beneficial in the practice of the disclosed methods. The kits can include any one or more of the vectors disclosed herein, along with other necessary or optional components. For example, the kits could include primers to perform the amplification reactions discussed in certain embodiments of the methods, as well as the buffers and enzymes required to use the primers as intended.

It is understood that the kit can contain a nucleic acid comprising in 5′ to 3′ order: a first minimal piggyBac inverted repeat element, a promoter (constitutive or inducible), a gene coding sequence (transcriptional unit) or region to receive a gene coding sequence (transcriptional unit), a IRES element, a second gene coding sequence or region to receive a gene coding sequence, and a second minimal piggyBac inverted repeat element.

In a further aspect, the kit can contain a nucleic acid comprising in 5′ to 3′ order: a first minimal piggyBac inverted repeat element, a promoter (constitutive or inducible), a gene coding sequence (transcriptional unit) or region to receive a gene coding sequence sequence (transcriptional unit), a IRES element, a second gene coding sequence or region to receive a gene coding sequence, a second promoter (constitutive or inducible), a separate and third gene sequence, and a second minimal piggyBac inverted repeat element.

In a further aspect, the kit can contain a nucleic acid comprising in 5′ to 3′ order: a first minimal piggyBac inverted repeat element, a promoter (constitutive or inducible), a gene coding sequence (transcriptional unit) or region to receive a gene coding sequence (transcriptional unit), a second promoter (same or different, constitutive or inducible), a second gene coding sequence or region to receive a gene coding sequence, and a second minimal piggyBac inverted repeat element.

10. Compositions with Similar Functions

It is understood that the compositions disclosed herein have certain functions, such as directing a transposon to a target nucleic acid or binding to target nucleic acid. Disclosed herein are certain structural requirements for performing the disclosed functions, and it is understood that there are a variety of structures which can perform the same function which are related to the disclosed structures, and that these structures will ultimately achieve the same result.

### D. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning. A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System IPlus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process for Making the Compositions

Disclosed are processes for making the compositions as well as making the intermediates leading to the compositions. For example, disclosed are nucleic acids for the construction of a chimeric transposase obtained from linking a transposase [e.g. Tc1 (Reference No. NM_{—}061407, AI878683, AI878522, AI794017); P-element (Rio et al., Cell (1986) 44:21-32; among others)] to a DNA directing factor [e.g., LexA DBD (Accession No. J01643-V0029-V00300, Hin DNA binding domain (Reference No. J03245), STF-1 DNA binding domain (Reference No. S67435, corresponding to a.a. 140-215 described in Leonard et al. (1993) Mol. Endo. 7:1275-1283), among others]. The sequences of these and other known transposases can be obtained at Entrez Nucleotide Database, or GenBank or other nucleotide or protein search engines. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in a chimeric transposase obtained from linking a transposase [e.g. Tc1 (Reference No. NM_{—}061407, AI878683, AI878522, AI794017); P-element (Rio et al., Cell (1986) 44:21-32; and among others listed herein. The sequences can be obtained at Entrez Nucleotide Database, or GenBank or other nucleotide or protein search engines])] to a DNA directing factor [e.g., LexA DBD (Accession No. J01643-V0029-V00300, Hin DNA binding domain (Reference No. J03245), STF-1 DNA binding domain (Reference No. S67435, corresponding to a.a. 140-215 described in Leonard et al. (1993) Mol. Endo. 7:1275-1283), and among others listed herein. The sequences can be listed herein. The sequences can be obtained at Entrez Nucleotide Database, or GenBank or other nucleotide or protein search engines]] and a sequence controlling the expression of the nucleic acid.

Also disclosed are nucleic acid molecules produced by the process comprising linking in an operative way a nucleic acid molecule comprising a sequence having 80% identity to a sequence set forth in a chimeric transposase obtained from linking a transposase [e.g. Tc1 (Reference Nos. NM_{—}061407, AI878683, AI878522, AI794017); P-element (Rio et al., Cell (1986) 44:21-32; and among others listed herein. The sequences can be obtained at Entrez Nucleotide Database, or GenBank or other nucleotide or protein search engines])] to a DNA directing factor [e.g., LexA DBD (Accession No. J01643-V0029-V00300, Hin DNA binding domain (Reference No. J03245), STF-1 DNA binding domain (Reference No. S67435, corresponding to a.a. 140-215 described in Leonard et al. (1993) Mol. Endo. 7:1275-1283), and among others listed herein. The sequences can be obtained at Entrez Nucleotide Database, or GenBank or other nucleotide or protein search engines], and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence of a transposase set forth in a chimeric transposase obtained from linking a transposase [e.g. Tc1 (Reference Nos. NM_{—}061407, AI878683, AI878522, AI794017); P-element (Rio et al., Cell (1986) 44:21-32; and among others listed herein. The sequences can be obtained at Entrez Nucleotide Database, or GenBank or other nucleotide or protein search engines])] to a DNA directing factor [e.g., LexA DBD (Accession No. J01643-V0029-V00300, Hin DNA binding domain (Reference No. J03245), STF-1 DNA binding domain (Reference No. S67435, corresponding to a.a. 140-215 described in Leonard et al. (1993) Mol. Endo. 7:1275-1283), and among others listed herein. The sequences can be obtained at Entrez Nucleotide Database, or GenBank or other nucleotide or protein search engines] and a sequence controlling the expression of the nucleic acid.

Disclosed are nucleic acid molecules produced by the process comprising linking in an operative way a nucleic acid molecule comprising a comprising a sequence encoding a fusion polypeptide containing two DNA binding domains (or a DNA binding and a protein binding domain) [e.g., LexA DBD (Accession No. J01643-V0029-V00300, Hin DNA binding domain (Reference No. J03245) linked to the STF-1 DNA binding domain (Reference No. (Reference No. S67435, corresponding to a.a. 140-215 described in Leonard et al. (1993) Mol. Endo. 7:1275-1283) and among others listed herein which can be combined. The sequences can be obtained at Entrez Nucleotide Database, or GenBank or other nucleotide or protein search engines] and a sequence controlling an expression of the nucleic acid molecule.

Disclosed are nucleic acid molecules produced by the process comprising linking in an operative way a nucleic acid molecule comprising a sequence encoding a fusion polypeptide containing two DNA binding domains (or a DNA binding and a protein binding domain) [e.g., LexA DBD (Accession No. J01643-V0029-V00300, Hin DNA binding domain (Reference No. J03245) linked to the STF-1 DNA binding domain (Reference No. S67435, corresponding to a.a. 140-215 described in Leonard et al. (1993) Mol. Endo. 7:1275-1283) and among others listed herein which can be combined. The sequences can be obtained at Entrez Nucleotide Database, or GenBank or other nucleotide or protein search engines.] having 80% identity to a peptide and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### PiggyBac Transposon-Mediated Gene Transfer in Human Cells

Plasmid DNA. pCMV-SB, SB12 and pT3 have been described previously [4, 5, 10]. pBac[3XP3-EGFPafm] and the piggyBac transposase (“helper”) plasmid have been described previously [23, 24]. A kanamycin/neomycin resistance cassette was created by PCR from pIRES2-EGFP (Clonetech, Mountain View, Calif.) and subcloned into the BglII site of pBac[3XP3-EGFPafm] creating pPB-KN. The piggyBac helper plasmid was digested with BamHI followed by creation of blunt ends with Klenow and restriction digestion with SacII. This piggyBac transposase fragment was then subcloned into SacII and PsiI digested pCMV-SB to create pCMV-piggyBac. To create pTpB, PCR was used to replace the left IR (LIR) element of pT3 with the minimal 311 bp LIR of piggyBac and the right IR (RIR) of pT3 was replaced with the minimal 235 bp RIR of piggyBac [18]. Combination “helper-independent” transposase-independent”transposase-transposon vectors were generated by digesting the appropriate transposase plasmid (pCMV-SB12 or pCMV-piggyBac) with SbfI and then subcloning the transposase containing fragment into a unique SbfI site in the corresponding transposon plasmids (pT3 or pTpB). All plasmid constructs were confirmed by restriction digestion and DNA sequencing.

Transposition assays. HEK-293 or HeLa cells (1×10⁶) were transiently transfected with plasmid DNA using FuGENE®6 (Roche Diagnostics, Indianapolis, Ind.). Two days post transfection, cells were split to various densities (1:60 or 1:600 dilution) and placed in media containing 800 μg/mL G418. After 2 weeks of G418 selection, colonies of cells were fixed in 10% formaldehyde/phosphate buffered saline (PBS), stained with methylene blue in PBS and counted [1, 10]. For overproduction inhibition assays, nonrecombinant pIRESpuro3 vector was used to equalize the total DNA amount in each transfection. Transfection efficiency of 50% was routinely observed for HeLa and HEK-293 cells using FuGENE®6 and plasmid encoding green fluorescent protein (GFP). This level of transfection efficiency in combination with the assumption of 100% plating efficiency, quantification of the number of cells transfected, and the use of cell dilutions as outlined above was used to estimate the yield of cells having undergone transposition. We then used G418-resistant colony counts as a proxy for transposition activity. Multiple experimental repetitions were always performed using separate transfections on different days with 2 or more individual preparations of DNA.

Excision assays. HEK-293 cells were transiently transfected with separate plasmids containing transposase (400 ng) and transposon (2 μg) plasmids using FuGENE®6. Three days post-transfection (after excision and plasmid repair have occurred), plasmid DNA was isolated using a QiaPrep spin column (Qiagen, Valencia, Calif.). Isolated plasmid DNA was then used as a template for a PCR reaction designed to amplify from plasmids that have undergone excision of the transposon followed by repair of the remaining vector DNA [10, 25]. A population of PCR products from two different transfections was gel purified, subcloned into the TOPO 2.1 vector (Invitrogen, Carlsbad, Calif.) and used to transform bacteria. Plasmid DNA from isolated bacterial colonies was sequenced using a T7 primer to determine the DNA sequence remaining after excision and repair.

Western analysis. Three days after transfection, cells were lysed as described previously [10]. Total protein was quantified using Bradford analysis. Protein (15 μg per lane) of was loaded onto precast 10% polyacrylamide gels (Biorad, Hercules, Calif.) and subjected to subjected to SDS-PAGE. Gels were transferred to nitrocellulose and immunoblotted using polyclonal anti-SB antibodies (kindly provided by Perry Hackett) or monoclonal anti-HA antibodies as described previously [2, 10].

Plasmid rescue of genomic integration sites. To determine integration sites in cultured cells, we modified a protocol from Yant, et al. [26] Cultured HEK-293 or Hela cells were transfected with pTpB (2 μg) and pCMV-piggyBac (1 μg) using FuGENE®6. After 2-3 weeks of G418 selection, genomic DNA was isolated from a near confluent 100 mm dish of cells. DNA was then treated with NdeI and shrimp alkaline phosphatase to reduce transposon plasmid background (NdeI cuts within the plasmid backbone but outside of the transposon segment). DNA was then digested with NheI, SpeI, and XbaI which do not cut within the transposon segment but do create compatible cohesive ends. Self-ligation was performed using T4 DNA ligase. DH10B E. Coli were transformed by electroporation and subsequently plated on LB-agar with kanamycin for selection. Kanamycin resistant colonies were replica plated on LB-ampicillin plates. Colonies that grew in the presence of kanamycin but not in the presence of ampicillin (pTpB backbone harbors ampicillin resistance) were presumed to represent cells with transposon integrations. We isolated plasmid DNA and performed sequencing using a primer which reads through the 5′ IR element of the piggyBac transposon (5′-TTCCACACCCTAACTGACAC-3′).

Mapping of genomic integration sites. The UC Santa Cruz BLAT genome web-browser (human, March 2006 assembly) was used to map piggyBac integration sites in the human genome. We used ˜80 bp of high quality sequence starting immediately after the terminal TTAA in the IR element of the transposon segment for BLAT searches. We determined sequences to consist of true piggyBac integration sites if 1) genomic sequence began immediately after the terminal transposon TTAA, 2) mapping of the genomic integration site revealed an intact immediate upstream TTAA target site where the integration occurred, and 3) the DNA sequence was high quality and matched only one genomic locus with >95% identity. Of the 672 total sequences evaluated, we were able to unambiguously assign 575 integration sites (320 in HEK-293 and 255 in Hela cells) to single genomic loci within the human genome of which all were unique (i.e., no locus was hit in both HEK-293 and Hela cells). The remaining sequences were either unreadable or mapped to more than one genomic locus. We were unable to recover any inter-plasmid transposition events in our cultured cells which had been under selection for 2-3 weeks.

The site of genomic integration was evaluated for RefSeq genes, CpG islands, islands, transcriptional start sites, and repeat elements such as long interspersed nuclear elements (LINE), short interspersed nuclear elements (SINE), long terminal repeats (LTR), DNA elements, and microsatellite repeats. Integration into a RefSeq gene was defined as occurring between the transcriptional start and stop sites of the gene. Chi square (x²) analysis was then used to compare the frequencies of piggyBac integrations into specific genomic elements to those previously reported for SB and 10,000 computer simulated random integration events [7] 282. Sequence logo analysis. Weblogo [27] was used to analyze piggyBac integration sites determined by our study to evaluate for consensus sequence motifs. The standard logo plot reveals a possible consensus sequence with the height of the nucleotide representing the level of conservation at that position. The logo frequency plot uses nucleotide height to represent the frequency of that nucleotide occurring at a given position within the integration target site sequence.

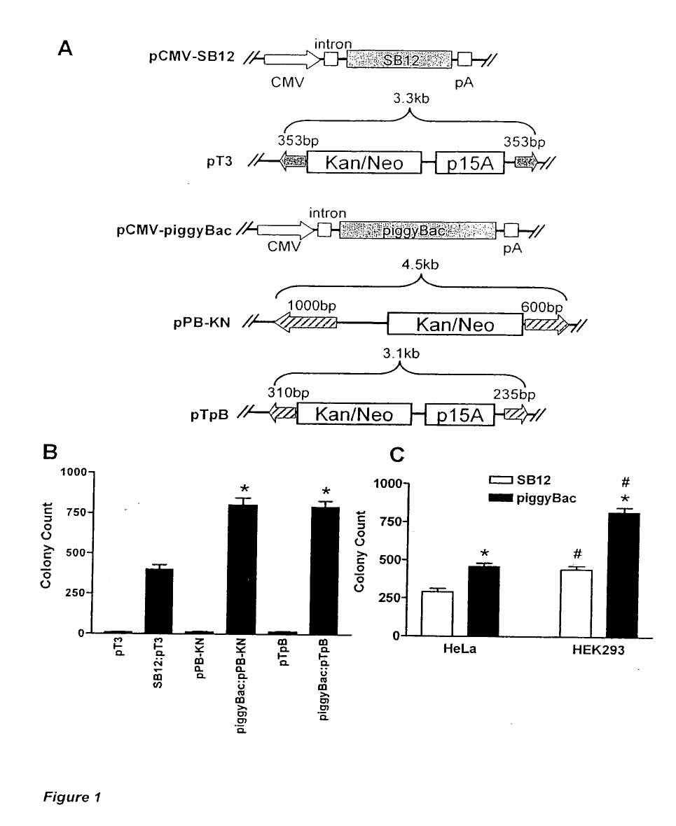

Efficient piggyBac-mediated transposition in human cells. To compare the transposition activities of piggyBac and SB in cultured human cells, the SB transposase cDNA was replaced with that of the piggyBac transposase in the pCMV-SB plasmid. This enables expression of both transposases from the same promoter in identical plasmid vectors (FIG. 1A). The original piggyBac transposon used for our experiments included a GFP transgene within the transposon interrupting the open reading frame of the piggyBac transposase (pBac[3XP3-EGFPafm]) [23, 24]. In order to quantify transposition in human cells, we inserted a kanamycin/neomycin resistance cassette into the piggyBac transposon, creating pPB-KN (FIG. 1A). A colony count assay of G418 resistant clones of HEK-293 cells was then used as a proxy for transposition activity to enable comparisons of this piggyBac transposon to that of a previously reported hyperactive SB variant (SB12) in combination with a hyperactive SB transposon (pT3). The combination of SB12 with the pT3 transposon increases SB transposition 2-4 fold over the native SB system [5, 10].

In our experiments the piggyBac transposon system exhibits ˜2-fold greater transposition activity in HEK-293 cells than the combination of SB12 with pT3 (FIG. 1B). A piggyBac transposon was also engineered that had the same plasmid backbone and transgene components as SB pT3 to exclude plasmid structure as a contributing factor to differences in transposition activity. Specifically, the IR elements of the SB pT3 transposon plasmid were replaced with minimal IR elements of the piggyBac transposon previously reported to exhibit high efficiency in insects [17, 18] (FIG. 1A). This piggyBac transposon, pTpB, showed no reduction in activity in HEK-293 cells when compared to the pPB-KN transposon with the full transposon terminal elements (FIG. 1B). These results demonstrate that the piggyBac transposon system has more transposition activity in HEK-293 cells than native and a previously engineered hyperactive SB. The maximal activity obtained with SB or piggyBac in both HEK-293 and HeLa cells was also compared using DNA amounts that achieved optimal transposon efficiency (400 ng of transposase and 2 μg of transposon). It was found that both transposon systems were more active in HEK-293 cells when compared to HeLa cells (FIG. 1C). Based on quantification of transfection efficiency with a GFP marker (data not shown), estimating the number of cells transfected, and using our colony count assays, we estimated that piggyBac transposition occurred in ˜10-15% of transfected HEK-293 cells. The integration frequency exhibited by piggyBac in HEK293 cells determined using Southern blot analysis appears to be very high (˜12-15 integrations per clone, data not shown).

PiggyBac excision is precise in human cells. Excision of SB transposons creates a predictable “footprint” mutation in the donor plasmid or in genomic DNA [25, 28, 29]. This footprint mutation includes 3 bp in addition to an added TA element creating a 5 bp insertion. By contrast, piggyBac excision in insects was previously found to lack footprint mutations [13, 30, 31] as evidenced by reconstitution of the TTAA target sequence frequently without insertion or deletion mutations. To examine this phenomenon in human cells, we performed excision site sequence analysis of piggyBac transposons in HEK-293 cells using a PCR based excision assay [10]. SB and piggyBac transposon excision events were detected only in the presence of their respective transposases (FIG. 2). Subcloned piggyBac PCR products resulting from excision were sequenced to evaluate piggyBac excision and repair. This analysis revealed reconstitution of the TTAA target sequence without insertions or deletions in 14 out of 15 subclones whereas one PCR product revealed a TTAA duplication. These results confirm that piggyBac excision frequently lacks footprint mutations in HEK293 cells consistent with what has previously been observed in insect cells.

PiggyBac integration into the human genome. Although piggyBac integration has been characterized in insects, the integration site-specificity for intragenic and intergenic elements within the human genome is not well known. Other transposon systems such as SB exhibit some degree of integration site preferences that differ among species [7]. To date, only 18 human genomic integration sites have been reported for piggyBac [20]. We performed 2 performed 2 separate transfections of HEK-293 and Hela cells (4 transfections total) and used plasmid rescue of integration sites to create 4 separate piggyBac integration libraries. Sequencing combined with computational analyses was used to successfully map 575 piggyBac transposon integration sites into the human genome.

The frequency of piggyBac integrations into known genomic elements was analyzed (Table 1). Although our analysis may be biased by evaluating integration sites after selection, our approach is comparable to the previously reported integration sites for SB in human cells which were also obtained under selection [7]. PiggyBac demonstrated a slightly higher frequency of integrations into RefSeq annotated genes than that previously reported for SB or randomly generated integration sites, but a lower frequency than that reported for HIV-1 [7]. Interestingly, piggyBac exhibited a bias toward a 10 kb window around known transcriptional start sites. Five piggyBac integrations into exons were observed, but all were in 5′ or 3′ untranslated regions. Our analysis of piggyBac integration into genomic repeat elements revealed a preference for LTRs, a noted difference from SB [7] (Table 2). A lack of piggyBac integration into microsatellite repeat elements was observed, which was a previously reported bias observed for SB in human cells. From these data it was concluded that piggyBac exhibits different genomic integration site-selectivity as compared with SB and HIV-1.

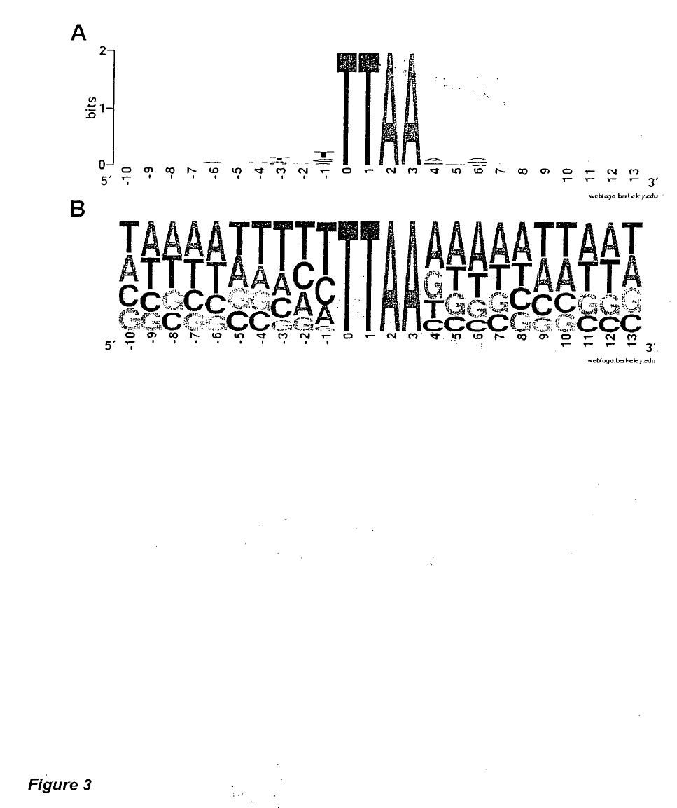

Sequence logo analysis was used to evaluate piggyBac integration sites in the human genome to ascertain the existence of consensus integration flanking sequences (see supplementary data S1). SB integration has been shown to occur at TA dinucleotides with a surrounding palindromic consensus sequence [7, 32-34]. In contrast to SB, sequence logo analysis of 575 piggyBac integration sites ascertained from human cells revealed no obvious consensus sequence (FIG. 3A), other than the required TTAA tetranucleotide integration sequence, and this is consistent with prior observations made in a variety of insect species [18, 34]. However, a nucleotide frequency plot of integrations for piggyBac did reveal a palindromic “preference” for upstream and downstream repetitive A or T sequences surrounding the central TTAA nucleotide element (FIG. 3B, data supplement S2). Therefore, piggyBac apparently preferentially targets palindromic TA rich sequences in the human genome that are different from that of SB.

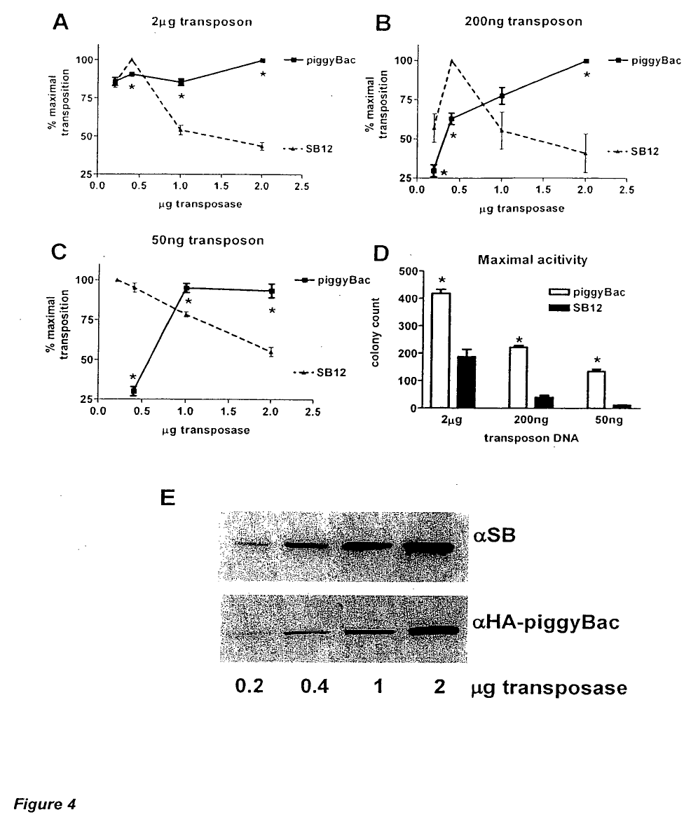

PiggyBac lacks overproduction inhibition. A known phenomenon of the SB transposon system is overproduction inhibition which occurs with increasing transposase expression. This can be detrimental for in vitro and in vivo gene transfer and occurs with both both the native SB transposase and hyperactive variants [3, 5, 10-12]. The transposition activity in cultured human cells transiently transfected was compared with either piggyBac to SB using 2 μg, 200 ng, and 50 ng of transposon DNA while varying the amount of transposase plasmids. For these experiments, the recombinant piggyBac transposase/transposon plasmids were used that differ from the SB constructs only by the piggyBac cDNA and IR elements (FIG. 4A-C). At all three transposon DNA amounts, it was observed that overproduction inhibition with SB12 manifested as decreased G418 resistant colony formation with higher amounts of transfected transposase plasmid. By contrast, piggyBac did not demonstrate overproduction inhibition at any of the three transposon DNA levels even when the molar transposase-to-transposon ratio was 43:1 (50 ng transposon and 2 μg of transposase, equivalent to a molar ratio of 50:1 for SB12). When comparing maximal activity of the two systems at the three different transposon DNA amounts, we observed piggyBac to be 2-10 fold more active than the SB12/pT3 combination (FIG. 4D). FIGS. 4A-C therefore represent how the maximal colony counts obtained in FIG. 4D were affected by varying the amount of transposase transfected for both SB12 and piggyBac.

Western analysis of transposase expression was performed to verify that increased SB transposase expression correlated with decreased transposition (i.e. overproduction inhibition), and that piggyBac transposase expression was increased with increasing transfected amounts without loss of transposition activity (i.e. no overproduction inhibition). Immunoblot analysis of transfected cell lysates using polyclonal anti-SB antibodies confirmed increased SB transposase expression with increased transfected transposase plasmid DNA (FIG. 4E). A hemagglutinin (HA) epitope tag was added to the N-terminus of the piggyBac transposase and demonstrated no effect on transposition activity compared to the native enzyme (data not shown). Western analysis with monoclonal anti-HA antibodies revealed that expression of piggyBac transposase protein increased in proportion to the amount of transfected DNA. These findings indicate that piggyBac transposition activity is not affected by overproduction inhibition within the wide variety of ranges tested (FIG. 4E).

Combination piggyBac vectors with increased activity in human cells. Combined SB transposase-transposon vectors (referred to as “helper-independent”) have previously been generated [12]. Due to overproduction inhibition, promoter strength was of great importance in mediating the amount of gene transfer in vivo with strong promoters such as the immediate early promoter of CMV resulting in less transposition than weaker promoters. As promoters. As piggyBac lacks overproduction inhibition, this system is more amenable to generating helper-independent vectors encoding transposase and transposon in the same plasmid. Such vectors facilitate gene transfer in vivo as cells would only require transfection with one plasmid instead of two separate transposase and transposon vectors.

Helper-independent SB12/pT3 and piggyBac transposase-transposon plasmids were engineered using the strong CMV immediate early promoter to drive expression of the transposase and compared transposition activity in HEK-293 cells (FIG. 5A). Transposition resultant from supplying the transposase and transposon plasmids separately (1:1 molar ratio) was compared to that of the helper-independent plasmid while keeping the total DNA quantity constant in all transfections (FIG. 5B). For SB transposition there was a trend toward reduced activity using the helper-independent vector. Although the CMV promoter is not optimal for SB in a cis vector formulation [12], we utilized this strong promoter to exaggerate the possibility of overproduction inhibition. Using the CMV promoter to drive transposase expression in a combined transposase-transposon plasmid, piggyBac activity was 2-fold greater in cells transfected with the combined piggyBac vector as compared to separate piggyBac plasmids. This observation is explainable by the lack of overproduction inhibition and our presumption that cells transfected with the combination plasmid expressed piggyBac transposase in the presence of transposon DNA at a higher frequency.

### a) Example 2

### PiggyBac Mediated Multi-Gene Integration In Vitro and In Vivo

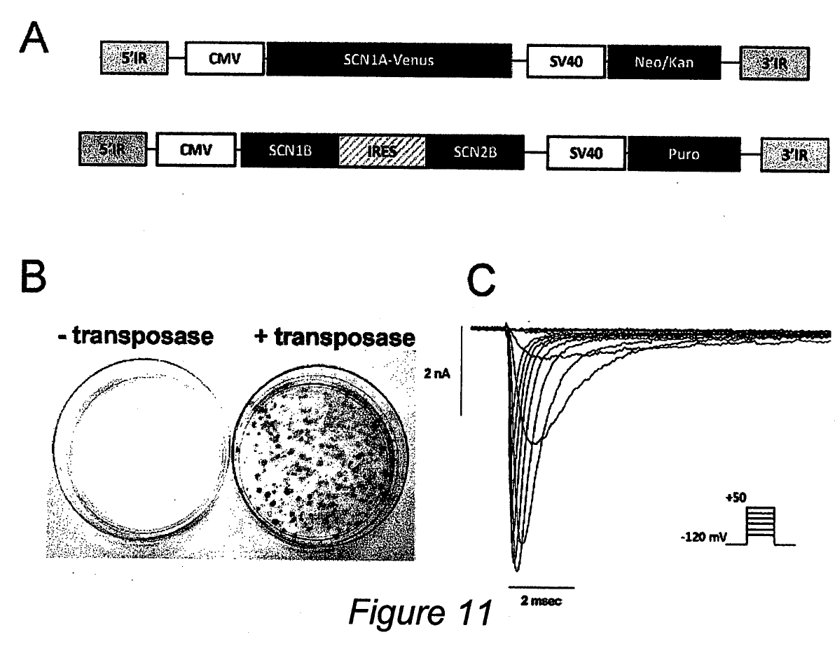

Plasmids with multi-transgene transposons were constructed using standard recombinant DNA methods. All plasmid constructs were confirmed by restriction digestion and DNA sequencing. HEK-293 cells were grown in Dulbecco's Modified Eagle's Medium supplemented with 10% fetal bovine serum (Atlanta Biologicals, Norcross, Ga.), L-glutamine (2 mM) and penicillin-streptomycin (50 units/ml and 50 μg/ml, respectively) in a humidified, 5% CO₂ atmosphere at 37° C. Cells were co-transfected with both transposon plasmids illustrated in panel A (FIG. 11) with (+transposase) or without (-transposase) a plasmid encoding the piggyBac transposase (pCMV-piggyBac) using FuGENE 6 (Roche Applied Science). Seventy-two hours after transfection, the cells were passaged and placed under dual selection with puromycin (3 ug/mL) and G418 (800 ug/mL) for 3 weeks. One set of puromycin/G418 resistant cells were stained with methylene blue to count colonies as a quantitative measure of stable integration efficiency (FIG. 11). Clonal populations of puromycin/G418 resistant cells were isolated using puromycin/G418 resistant cells were isolated using 3 mm cloning disks. The isolated clones were expanded in culture for 3 weeks under puromycin/G418 selection and frozen for storage using standard cryo-protective methods. An aliquot of selected cells were plated for use in electrophysiology experiments to screen for stable expression of the genes encoded on each transposon.

Sodium channel currents were recorded at room temperature using the whole-cell patch clamp method. Patch pipettes were fabricated from borosilicate glass (Warner Instrument Co., Hamden, Conn., U.S.A) by a multistage P-97 Flaming-Brown micropipette puller (Sutter Instruments Co., San Rafael, Calif., U.S.A.) and fire-polished by using a microforge (MF 830, Narashige, Japan). Pipette resistance was between 1.0 and 2.0 MΩ. The pipette solution consisted of (in mM) 110 CsF, 10 NaF, 20 CsCl, 2 EGTA, 10 HEPES, with a pH of 7.35 and osmolarity of 310 mOsmol/kg. The bath solution contained in (mM): 145 NaCl, 4 KCl, 1.8 CaCl₂, 1 MgCl₂, 10 HEPES, with a pH of 7.35 and osmolarity of 310 mOsmol/kg. The osmolarity was adjusted with sucrose. The bath solution was continuously exchanged by a gravity-driven perfusion system. The reference electrode consisted of a 2% agar bridge with composition similar to the bath solution. Cells were allowed to stabilize for 10 min after establishment of the whole-cell configuration before current was measured (FIG. 11).

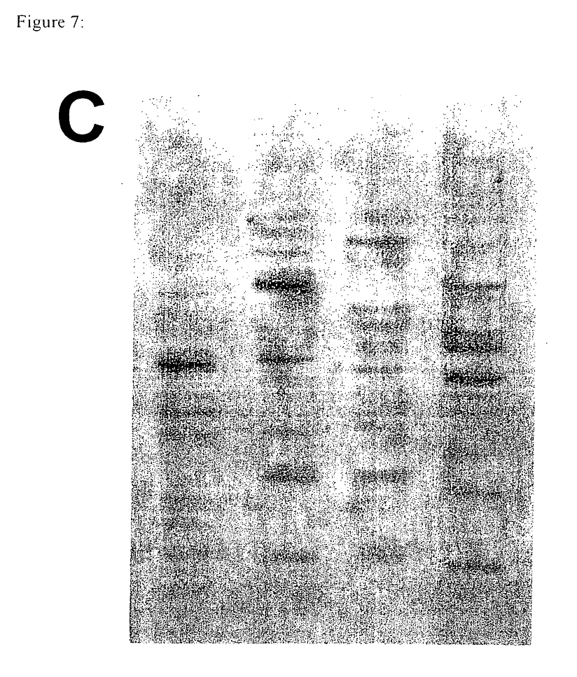

PiggyBac is uniquely capable of delivering multiple different transgenes into the genome of a cell of interest. Our data indicate that piggyBac is capable of integrating >15 different transposons in a given cell (FIG. 7). Given that piggyBac is capable of delivering large transposons containing 10-15 kb without loss of activity, genetic engineering with multiple or even multiple large genes can be realized. The data demonstrate integration of two transposons of size 5.8 kb and 10.9 kb (FIG. 11).

PiggyBac can be harnessed to deliver multiple transgenes at once both in vitro and in vivo. In HEK293 cells, we estimate that transposition occurs in ˜50% or more of cells transfected with >15 transposons integrated per cell. One could therefore simultaneously co-transfect transposons harboring different genes of interest with confidence that some of the cells would integrate multiple different gene harboring transposons. The data demonstrate successful stable integration of 5 different transgenes into the same cell. The genes include three voltage-gated sodium channel subunits (SCN1A-Venus, SCN1B, SCN2B) and two antibiotic resistance genes (Neo/Kan conferring resistance to aminoglycoside antibiotics neomycin, kanamycin and G418; and Puro conferring resistance to the antibiotic puromycin). Moreover, one of the sodium to the antibiotic puromycin). Moreover, one of the sodium channel genes is actually a fusion of the channel coding region for SCN1A and the fluorescent protein Venus, so one could actually consider this experiment to include 6 transgenes (FIG. 11).

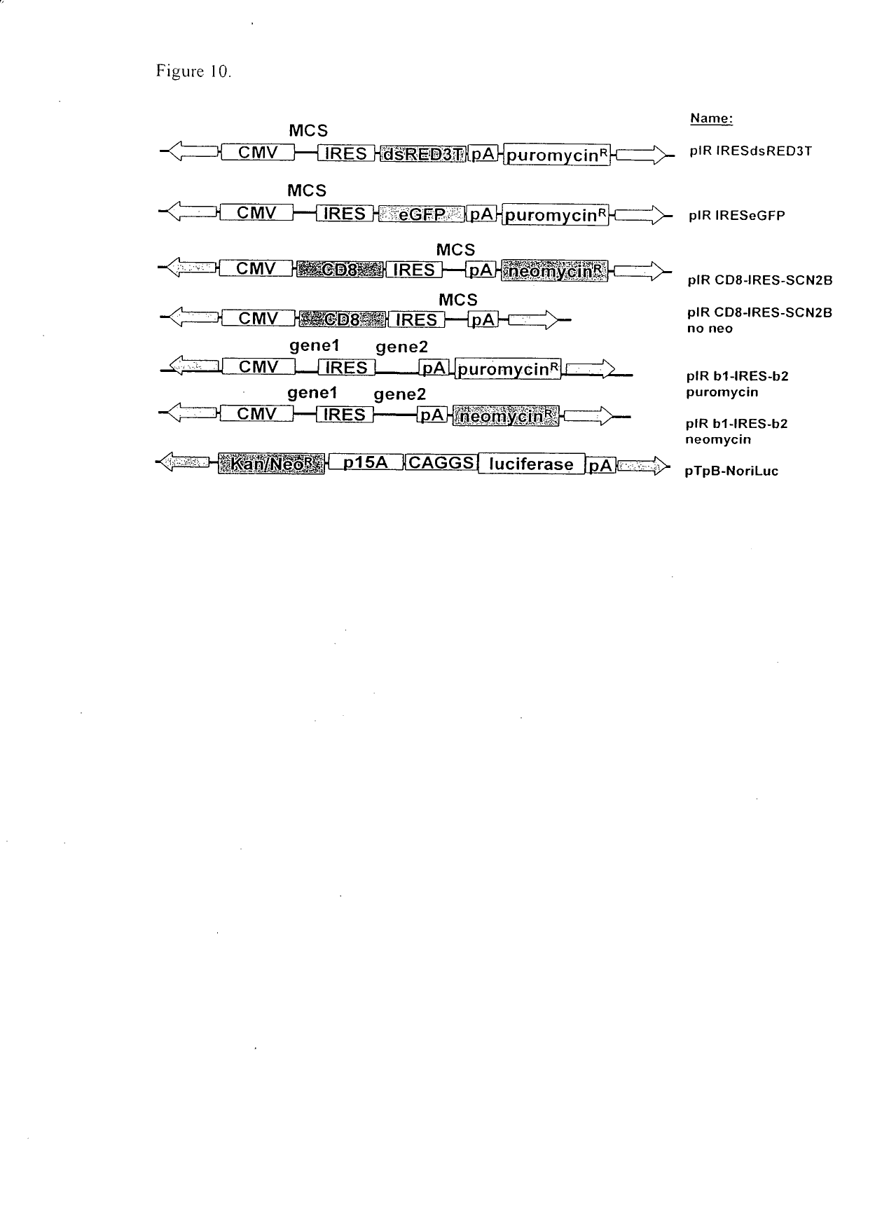

Transposons containing multiple different selectable markers were generated for selection of cells expressing genes from the transposons of interest. In doing so, transposons were created using IRES vectors harboring eGFP, dsRED, CD8, puromycin, neomycin, and luciferase all in bicistronic or multi-gene vectors (FIG. 5). This permits analysis and selection of cells which have taken up the various transposons of interest. The pIR constructs in FIG. 10 can be used for this purpose, wherein the first and second genes are expressed on a single mRNA, wherein the two open reading frames (ORFs) are separated by an internal ribosome entry site (IRES) that allows for initiation of translation in the middle of an mRNA.

Selection of cells having integrated multiple different piggyBac transposons The development of piggyBac transposons with a variety of selectable markers permits the selection of cell populations with multiple transgenes of interest. The Baylor Cytometry and Cell Sorting Core is capable of sorting 13 different spectral colors into wells of cell culture plates. This permits investigators to place their gene of interest into the first slot of our IRES selectable transposon vectors. Cells expressing the transgene of interest can then be selected out using fluorescence activated cell sorting (FACS) into culture plates. The capability to sort multiple different colors (and therefore multiple different transgenes) simultaneously will permit selection of cells which contain multiple transgenes stably integrated into one cell which can then be expanded to a clonal population. FACS analysis will permit determination of how many different transgenes can be integrated into a cell type of interest. Evaluation of cell types commonly used for drug discovery applications such as HEK293 and Chinese hamster ovary (CHO) cells as well as cell types used for in vivo cell therapies such as stem cells and cytotoxic T cells can be conducted. The data demonstrate an additional selection strategy for selecting cells transfected with multiple transgenes utilizing antibiotic resistance (FIG. 11).

Using piggyBac to achieve regulatable gene expression in vivo Current gene transfer technology delivers one therapeutic gene with constitutive expression. This is not suitable for such disorders as growth hormone deficiency or erythropoietin deficiency (anemia) which would require the ability to not only turn the expression of the therapeutic gene on, but also off at given time points. The ability to deliver multiple transgenes simultaneously with the simultaneously with the use of an inducible promoter driving the therapeutic transgene of interest has the ability to overcome this obstacle.

Regulatable gene expression in vivo can be achieved using piggyBac. To further confirm this outcome we can deliver two different gene carrying transposons simultaneously to the liver of mice using hydrodynamic tail vein injection, a standardized in vivo gene delivery method which is touted to be able to deliver plasmid DNA to ˜50% of hepatocytes. One transposon carries luciferase with a tetracycline responsive promoter. The other transposon harbors the tetracycline responsive activator. In cells taking up both transposons, tetracycline treatment should permit luciferase expression. Genes can be delivered with and without transposase to verify that simultaneous transposition of two different transgenes can occur within the liver in vivo. The ability to simultaneously deliver multiple therapeutic genes provides therapy for multigenic disorders.

Using piggyBac to create stable cell lines for drug discovery applications The ability to simultaneously deliver multiple genes makes feasible the possibility of engineering cell lines for a wide variety of applications. A notable example relates to drug discovery. Stable cell lines could be generated which reconstitute signaling mechanisms at which drugs can be targeted to alter these processes. There are many cellular processes which currently cannot be evaluated for drug discovery due to the lack of stable cell lines. For instance, one cannot evaluate a variety of cell surface receptors as drug targets due to a lack of suitable cell lines expressing these receptors and the necessary down-stream signaling molecules to evaluate receptor signaling. The data demonstrate the generation of a stable cell line expressing three human voltage-gated sodium channel subunits and expression of a very robust sodium current in the cells (FIG. 11, panel C).

PiggyBac is uniquely capable of simultaneous stable delivery of multiple genes in vivo. This innovative approach allows not only for stable cell line generation but also for drug discovery applications, genetic engineering, and the ability to regulate transgene expression in vivo.

Another strategy can involves using multiple transposon systems. The Sleeping Beauty transposon system and the phiC31 integrase system can be used to non-virally deliver genes to cultured cell types of interest. All of these systems are efficient at integrating transgenes into the genomes of cells. Multiple rounds of delivery such as piggyBac followed by SB and then phiC31 can also achieve the same end products. As these systems do not have cross reactivity, the genes integrated with the preceding delivery system will not be remobilized using will not be remobilized using the subsequent systems.

Nucleofection (Amaxa, Inc.) is a relatively recent and standard way of delivering DNA to a wide variety of difficult to transfect cell lines.

### F. REFERENCES

1. Ivics, Z. et al. (1997) Molecular reconstruction of Sleeping beauty, a Tc1-like transposon from fish, and its transposition in human cells. Cell 91: 501-510.
2. Baus, J. et al. (2005) Hyperactive transposase mutants of the Sleeping Beauty transposon. Mol. Ther. 12: 1148-1156.
3. Geurts, A. M. et al. (2003) Gene transfer into genomes of human cells by the sleeping beauty transposon system. Mol. Ther. 8: 108-117.
4. Yant, S. R. et al. (2004) Mutational analysis of the N-terminal DNA-binding domain of Sleeping Beauty transposase: Critical residues for DNA binding and hyperactivity in mammalian cells. Mol. Cell. Bio. 24: 9239-9247.
5. Zayed, H. et al. (2004) Development of hyperactive Sleeping Beauty transposon vectors by mutational analysis. Mol. Ther. 9: 292-304.
6. Wilber, A. et al. (2006) RNA as a source of transposase for sleeping beauty-mediated gene insertion and expression in somatic cells and tissues. Mol. Ther. 13: 625-630.
7. Yant, S. R. et al. (2005) High-resolution genome-wide mapping of transposon integration in mammals. Mol. Cell. Bio. 25: 2085-2094.
8. Lampe, D. J., Grant, T. E., and Robertson, H. M. (1998) Factors affecting transposition of the Himar1 mariner transposon in vitro. Genetics 149: 179-187.
9. Lohe, A. R. and Hartl, D. L. (1996) Autoregulation of mariner transposase activity by overproduction and dominant-negative complementation. Mol. Biol. Evol. 13: 549-555.
10. Wilson, M. H., Kaminski, J. M., and George, A. L., Jr. (2005) Functional zinc finger/sleeping beauty transposase chimeras exhibit attenuated overproduction inhibition. FEBS Lett. 579: 6205-6209.
11. Converse, A. D. et al. (2004) Counterselection and co-delivery of transposon and transposase functions for Sleeping Beauty-mediated transposition in cultured mammalian cells. Biosci. Rep. 24: 577-594.
12. Mikkelsen, J. G. et al. (2003) Helper-independent Sleeping Beauty transposon-transposase vectors for efficient nonviral gene delivery and persistent gene expression in vivo. Mol. Ther. 8: 654-665.
13. Fraser, M. J. et al. (1996) Precise excision of TTAA-specific lepidopteran transposons piggyBac (IFP2) and tagalong (TFP3) from the baculovirus genome in cell lines from two species of Lepidoptera. Insect Mol. Biol. 5: 141-151.
14. Cary, L. C. et al. (1989) Transposon mutagenesis of baculoviruses: analysis of Trichoplusia ni transposon IFP2 insertions within the FP-locus of nuclear polyhedrosis viruses. Virology 172: 156-169.
15. Fraser, M. J. et al. (1995) Assay for movement of Lepidopteran transposon IFP2 in insect cells using a baculovirus genome as a target DNA. Virology 211: 397-407.
16. Elick, T. A., Lobo, N., and Fraser, M. J., Jr. (1997) Analysis of the cis-acting DNA elements required for piggyBac transposable element excision. Mol. Gen. Genet. 255: 605-610.
17. Li, X. et al. (2001) The minimum internal and external sequence requirements for transposition of the eukaryotic transformation vector piggyBac. Mol. Genet. Genomics 266: 190-198.
18. Li, X. et al. (2005) piggyBac internal sequences are necessary for efficient transformation of target genomes. Insect Mol. Biol. 14: 17-30.
19. Bauser, C. A., Elick, T. A., and Fraser, M. J. (1999) Proteins from nuclear extracts of two lepidopteran cell lines recognize the ends of TTAA-specific transposons piggyBac and tagalong. Insect Mol. Biol. 8: 223-230.
20. Ding, S. et al. (2005) Efficient transposition of the piggyBac (PB) transposon in mammalian cells and mice. Cell 122: 473-483.
21. Izsvak, Z., Ivics, Z., and Plasterk, R. H. (2000) Sleeping Beauty, a wide host-range transposon vector for genetic transformation in vertebrates. J. Mol. Bio. 302: 93-102.
22. Karsi, A. et al. (2001) Effects of insert size on transposition efficiency of the Sleeping Beauty transposon in mouse cells. Marine Biotechnology 3: 241-245.
23. Mohammed, A. and Coates, C. J. (2004) Promoter and piggyBac activities within embryos of the potato tuber moth, Phthorimaea operculella, Zeller (Lepidoptera: Gelechiidae). Gene 342: 293-301.
24. Thomas, J. L. et al. (2002) 3XP3-EGFP marker facilitates screening for transgenic silkworm Bombyx mori L. from the embryonic stage onwards. Insect Biochem. Mol. Biol. 32: 247-253.
25. Liu, G. Y. et al. (2004) Excision of Sleeping Beauty transposons: parameters and applications to gene therapy. J. Gene Med. 6: 574-583.
26. Yant, S. R. et al. (2000) Somatic integration and long-term transgene expression in normal and haemophilic mice using a DNA transposon system. Nat. Genet. 25: 35-41.
27. Crooks, G. E. et al. (2004) WebLogo: a sequence logo generator. Genome Res. 14: 1188-1190.
28. Izsvak, Z. et al. (2004) Healing the wounds inflicted by Sleeping Beauty transposition by double-strand break repair in mammalian somatic cells. Mol. Cell 13: 279-290.
29. Yant, S. R. and Kay, M. A. (2003) Nonhomologous-end-joining factors regulate DNA repair fidelity during Sleeping Beauty element transposition in mammalian cells. Mol. Cell. Bio. 23: 8505-8518.
30. Elick, T. A., Bauser, C. A., and Fraser, M. J. (1996) Excision of the piggyBac transposable element in vitro is a precise event that is enhanced by the expression of its encoded transposase. Genetica 98: 33-41.
31. Thibault, S. T. et al. (2004) A complementary transposon tool kit for Drosophila melanogaster using P and piggyBac. Nat. Genet. 36: 283-287.
32. Liu, G. et al. (2005) Target-site preferences of Sleeping Beauty transposons. J. Mol. Biol. 346: 161-173.
33. Vigdal, T. J. et al. (2002) Common physical properties of DNA affecting target site selection of Sleeping Beauty and other Tc1/mariner transposable elements. J. Mol. Biol. 323: 441-452.
34. Geurts, A. M. et al. (2006) Structure-based prediction of insertion-site preferences of transposons into chromosomes. Nucleic Acids Res. 34: 2803-2811.
35. Maragathavally, K. J., Kaminski, J. M., and Coates, C. J. (2006) Chimeric Mos1 and piggyBac transposases result in site-directed integration. Faseb J. online, Jul. 28, 2006
36. Narezkina, A. et al. (2004) Genome-wide analyses of avian sarcoma virus integration sites. J. Virol. 78: 11656-11663.
37. Schroder, A. R. W. et al. (2002) HIV-1 integration in the human genome favors active genes and local hotspots. Cell 110: 521-529.

### G. SEQUENCES

| | | | |
|---|---|---|---|
| Start | end | name | description |

SEQ ID No: 1 [pTpB]
Features:

| | | | |
|---|---|---|---|
| 402 | 712 | 5′IR | 5′IR |
| 1414 | 2197 | Kan/Neo | antibiotic resistance |
| 2577 | 3014 | p15A ori | ori of replication |
| 3305 | 3540 | 3′IR | 3′IR |
| 3971 | 4584 | pUC | pUC ori |
| 5761 | 4741 | b-lactamase | antibiotic resistance |

SEQ ID No: 2 [pCMV piggyBac]
Features:

| | | |
|---|---|---|
| 27 | 548 | CMV IE promoter |
| 672 | 768 | intron (SV40) |
| 862 | 2610 | piggyBac transposase |
| 2671 | | poly A |
| 3806 | 3163 | pUC ori |
| 4884 | 3954 | Amp R |

SEQ ID No: 3 [pPB-Nori]
Features:

| | | |
|---|---|---|
| 402 | 712 | 5′IR |
| 1414 | 2197 | Kan/Neo |
| 2577 | 3014 | p14A ori |
| 3304 | 3540 | 3′IR |
| 3653 | | poly A (complementary strand) |
| 5498 | 3714 | piggyBac transposase (complementary) |
| 5652 | 5556 | SV40 intron (complementary) |
| 6297 | 5776 | CMV immediate early promoter |
| 8418 | 7488 | b-lactamase |

SEQ ID No: 4 [piggyBac minimal 5′ IR]
SEQ ID No: 5 [piggyBac minimal 3′ IR]
SEQ ID No: 6 [humanized piggyBac]

| | | | |
|---|---|---|---|
| Start | end | name | description |

SEQ ID NO: 7
[multi-gene vectors: pIR IRESdsRED3T]
Features:

| | | |
|---|---|---|
| 1 | 750 | CMV IE enhancer and |
| | | promoter |
| 890 | 1022 | IVIS |
| 1092 | 1143 | multiple cloning site |
| 1144 | 1728 | IRES |
| 1732 | 2409 | dsRED3T |
| 2448 | 2669 | SV40 poly A |
| 3283 | 4458 | puromycin R gene |
| 4624 | 4859 | 3′IR |
| 5112 | 5972 | Amp R gene |
| 6805 | 7115 | 5′IR |

SEQ ID NO: 8
[multi-gene vectors: pIR IRESeGFP]
Features:

| | | |
|---|---|---|
| 53 | 637 | IRES |
| 634 | 1360 | eGFP |
| 1401 | 1622 | SV40 poly A |
| 2236 | 3411 | puromycin R gene |
| 3577 | 3812 | 3′IR |
| 4065 | 4925 | Amp R gene |
| 5758 | 6068 | 5′IR element |
| 6075 | 6824 | CMV IE enhancer and |
| | | promoter |
| 6964 | 7096 | IVIS element |

SEQ ID NO: 9
[multi-gene vectors: pIR-CD8-IRES-SCN2B]
Features:

| | | |
|---|---|---|
| 1 | 96 | CMV IE enhancer |
| 97 | 750 | CMV promoter |
| 1101 | 1808 | CD8 gene |
| 1978 | 2558 | IRES element |
| 2592 | 3239 | SCN2E gene |
| 3279 | 3500 | SV40 poly A |
| 4114 | 5481 | Neomycin R gene |
| 5647 | 5882 | 3′IR element |
| 6135 | 6995 | Amp R |
| 7828 | 8138 | 5IR element |

SEQ ID NO: 10
[multi-gene vectors: pIR-CD8-IRES-SCN2B-no_neo]
Features:

| | | |
|---|---|---|
| 1 | 96 | CMV IE enhancer |
| 97 | 750 | CMV promoter |
| 1101 | 1808 | CD8 gene |
| 1978 | 2558 | IRES element |
| 2592 | 3239 | SCN2B gene |
| 3279 | 3500 | SV40 poly A |
| 5023 | 5258 | 3′IR element |
| 5511 | 6371 | Amp R |
| 7204 | 7514 | 5IR element |

SEQ ID NO: 11
[multi-gene vectors: pIR B1-IRES-B2 puro]
Features:

| | | |
|---|---|---|
| 1 | 96 | CMV IE enhancer |
| 97 | 750 | CMV promoter |
| 890 | 1022 | IVIS |
| 1097 | 1753 | SCN1B gene |
| 1782 | 2362 | IRES element |
| 2396 | 3043 | SCN2B gene |
| 3083 | 3304 | SV40 poly A |
| 3918 | 5093 | puromycin R gene |
| 5259 | 5494 | 3′IR element |
| 5747 | 6607 | Amp R |
| 7440 | 7750 | 5IR element |

SEQ ID NO: 12
[multi-gene vectors: pIR B1-IRES-B2 neo]
Features:

| | | |
|---|---|---|
| 1 | 96 | CMV IE enhancer |
| 97 | 750 | CMV promoter |
| 890 | 1022 | IVIS |
| 1097 | 1753 | SCN1B gene |
| 1782 | 2362 | IRES element |
| 2396 | 3043 | SCN2B gene |
| 3083 | 3304 | SV40 poly A |
| 3918 | 5285 | neomycin R gene |
| 5451 | 5686 | 3′IR element |
| 5939 | 6799 | Amp R |
| 7632 | 7942 | 5IR element |

SEQ ID NO: 13
[multi-gene vectors: pTpB-NoriLuc]
Features:

| | | |
|---|---|---|
| 402 | 712 | 5′IR |
| 1414 | 2197 | Kan/Neo R gene |
| 2577 | 3014 | p15A origin of replication |
| 3199 | 4826 | CAGGS promoter |
| 4893 | 6545 | luciferase (firefly) gene |
| 6577 | 6798 | SV40 poly A |
| 6815 | 7050 | 3′IR |
| 7481 | 8094 | pUC |
| 9181 | 8251 | Amp R gene |

SEQ ID NO: 14
[ZFP piggyBac vector sequence]
Features:

| | | |
|---|---|---|
| 232 | 820 | CMV promoter |
| 956 | 3379 | ZFP-piggyBac gene |
| 3517 | 3731 | BGH poly A |
| 4208 | 4580 | SV40 promoter |
| 4536 | 5327 | Neomycin R gene |
| 5382 | 5754 | SV40 poly A |
| 7695 | 6838 | Amp R gene |

What is claimed is:

1. A nucleic acid comprising a transcriptional unit or region to receive a transcriptional unit and an origin of replication functional in a target host cell flanked by minimal piggyBac inverted repeat elements.

2. The composition of claim 1 having the sequence as shown in SEQ ID NO:1.

3. The nucleic of claim 1 wherein the minimal piggyBac inverted repeat elements are 311 and 236 nucleotides in length.

4. The composition of claim 3, wherein the inverted repeats have the sequences as shown in SEQ ID NO: 4 and SEQ ID NO:5, respectively.

5. The nucleic acid of claim 1, wherein said transcriptional unit comprises a selectable marker coding sequence that encodes a polypeptide conferring antibiotic resistance linked to a promoter functional in a target organism, said antibiotic being selected from the group consisting of actinomycin, ampicillin, chloramphenicol, erythromycin, gentamycin sulfate, hygromycin, kanamycin, neomycin, penicillin, polymixin B sulfate and streptomycin sulfate.

6. A nucleic acid comprising the piggyBac transposase under the control of a CMV promoter with an intron between the two.

7. The composition of claim 6 having the sequence as shown in SEQ ID NO:2.

8. A nucleic acid comprising in 5′ to 3′ order: a CMV promoter, an intron, a piggyBac transposase coding sequence, a polyadenylation signal, a first minimal piggyBac inverted repeat element, a transcriptional unit or region to receive a transcriptional unit, an origin of replication functional in a target host cell, and a second minimal piggyBac inverted repeat element.

9. The composition of claim 8 having the sequence as shown in SEQ ID NO:3.

10. The composition of claim 8 wherein the piggyBac inverted repeat elements are 311 and 236 nucleotides in length.

11. A nucleic acid comprising multiple transcriptional units or a combination of transcriptional units and regions to receive a transcriptional unit or multiple regions to receive a transcriptional unit and that are together flanked by piggyBac inverted repeats, wherein each transcriptional unit or region to receive a transcriptional unit is separated from every other transcriptional unit or region to receive a transcriptional unit by an internal ribosome entry site, and operably linked to a promoter such that all transcriptional units are expressed via a bicistronic mRNA.

12. The nucleic acid of claim 11 having the sequences as shown in SEQ ID NO: 7, 8, 9, 10, 11 or 12.

13. A nucleic acid comprising in 5′ to 3′ order: a first minimal piggyBac inverted repeat element, a promoter, a transcriptional unit or region to receive a transcriptional unit, a second promoter, a second transcriptional unit or region to receive a transcriptional unit, and a second minimal piggyBac inverted repeat element.

14. The composition of claim 13 having the sequence as shown in SEQ ID NO:13.

15. A nucleic acid comprising in 5′ to 3′ order: a CMV promoter, a zinc finger-piggyBac chimeric transposase, a polyadenylation signal, an SV40 promoter, a neomycin gene, and a second polyadenylation signal.

16. The composition of claim 15, having the sequence as shown in SEQ ID NO: 14.

17. A nucleic acid that comprising the coding region of the piggyBac transposase that has been modified (humanized) at multiple nucleic acids

18. The composition of claim 17, wherein the modificed piggyBac transposase has the sequence as shown in SEQ ID NO:6.

19. A method delivering a transgene to a cell comprising transfecting or transforming the cell with a vector comprising the nucleic acid of claim 1 and a vector comprising a nucleic acid comprising the piggyBac transposase under the control of a CMV promoter with an intron between the two.

20. The method of claim 19 wherein more than three copies of a transgene are integrated per cell.

21. A method of delivering a transgene to a cell comprising transfecting or transforming the cell with a vector comprising nucleic acid comprising the piggyBac transposase under the control of a CMV promoter with an intron between the two and one or more vectors each comprising the nucleic acid of claim 11.

22. The method of claim 21 wherein more than three copies of a transgene are integrated per cell.

23. The method of claim 21 wherein two or more different transgenes are integrated per cell.

24. A method of delivering a transgene to a cell comprising transfecting or transforming cell with a vector comprising the nucleic acid of claim 8.

25. A method of overcoming overproduction inhibition by delivering a transgene to a cell comprising transfecting or transforming a vector or vectors according to the method of claim 19.

26. A method of overcoming overproduction inhibition by delivering a transgene to a cell comprising transfecting or transforming a vector or vectors according to the method of claim 21.

27. A method of overcoming overproduction inhibition by delivering a transgene to a cell comprising transfecting or transforming a vector or vectors according to the method of claim 24.

28. A method of maintaining piggyBac activity in a cell despite the covalent addition of a zinc finger DNA binding domain by delivering a transgene to a cell comprising transfecting or transforming a cell with a vector or vectors according to claim 15.
